(19) United States
(12) Patent Application Publication
(10) Pub. No.: US 2010/0112012 A1
(43) Pub. Date: May 6, 2010

(54) MODULATION OF IMMUNE RESPONSES

(75) Inventors: Andrei V. Gudkov, East Aurora, NY (US); Robert Fairchild, Mayfield Village, OH (US); Katerina Gurova, Orchard Park, NY (US)

Correspondence Address: POLSINELLI SHUGHART PC, 700 W. 47TH STREET, SUITE 1000, KANSAS CITY, MO, 64112-1802, US

(73) Assignee: CLEVELAND CLINIC FOUNDATION, Cleveland, OH (US)

(21) Appl. No.: 12/085,038

(22) PCT Filed: Nov. 14, 2006
(86) PCT No.: PCT/US2006/060864

§ 371 Date: Jan. 4, 2010

Related U.S. Application Data
(60) Provisional application No. 60/736,881, filed on Nov. 14, 2005.

(51) Int. Cl. A61K 31/473; A61K 39/00; A61P 31/00

(52) U.S. Cl. 424/278.1; 514/297

(57) ABSTRACT

Compositions and methods for modulating an NF-_{κ}B-mediated immune response are provided.

### CROSS-REFERENCE TO RELATED APPLICATIONS

This application is entitled to the benefit of U.S. Application No. 60/736,881, filed Nov. 14, 2005, the contents of which are incorporated herein by reference.

### FIELD OF THE INVENTION

The present invention relates to methods and compositions for modulating immune response in a mammal.

### BACKGROUND OF THE INVENTION

An immune response to an antigenic agent, be it a foreign antigen or an auto-antigen, is generally characterized by the production of antibodies by B lymphocytes and/or by destruction by T lymphocytes and/or natural killer (NK) cells of any cells displaying those antigens. Defects in B and/or T lymphoid cells, however, may result in the development of immunodeficiency diseases and/or the impairment of immune response function. The immune deficiency or defect may be congenital, caused by a mutation in a gene, or it may be acquired, for example, through a viral infection or as a result of the aging process. The thus produced defect may or may not be fatal, depending on the stage of stem cell or lymphocyte differentiation at which it occurs.

It is, therefore, highly desirable to identify relatively inexpensive, non-toxic, easily administered agents which are suitable for enhancing the immune response of mammals afflicted with an immunodeficiency disease(s), and/or for accelerating and enhancing the immune response of normal and elderly mammals when clinically indicated. It is also desirable in some instances to inhibit or suppress an immune response, such as in transplants, automimmune disease and allergic responses.

### SUMMARY OF THE INVENTION

A method of enhancing, inhibiting or suppressing a mammal's immune response is provided. The mammal may be administered an aminoacridine of the formula:

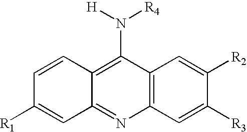

wherein,
R₁ is H or halogen;
R₂ is H or optionally substituted alkoxy;
R₃ is H or optionally substituted alkoxy; and
R₄ is H or optionally substituted aliphatic, aryl, or heterocycle.

The aminoacridine may be 9-aminoacridine or quinacrine. The mammal may be clinically normal, an immunodeficient subject, a subject afflicted by an auto-immune condition or a transplant subject. The aminoacridine may also be used boot the immune response of a mammal to a vaccine.

### BRIEF DESCRIPTION OF THE DRAWINGS

FIG. 1. Chemicals that activate p53 in RCC. A. Restoration of p53-mediated transactivation in RCC cells is accompanied by their death. p53-responsive reporter activity in RCC45ConALacZ cells (left panel, ONPG staining at 48 h) and cell survival after lentivirus-mediated transduction of p53 and GFP. B. Choice of readout cells and setting of selection criteria. MCF7 (a non-RCC control) and RCC cells, all containing the ConALacZ reporter, were treated with doxorubicin for 24 h (ONPG reaction normalized to protein concentration). C. Identification of 9-AA through chemical screening of compounds that activate p53 in RCC cells. 9AA derivatives (13 compounds, plus the original hit s30d9, 9AA, QC and m-AMSA were tested in a dose-dependent assay for activation of the p53 reporter in RCC45ConALacZ cells. Bars represent the relative activity of each compound, calculated as the maximal fold increase of p53 activation induced by the compound relative to the effect of 1 μM of doxorubicin (average of three experiments). D. Dose dependence of the effect of 9AA on p53 and p53-transcriptional targets, determined by the Western method in cells treated for 16 h with the indicated amounts of 9AA. E. 9AA activates p53 more strongly than doxorubicin in the majority of tumor cells tested. The p53-responsive reporter activity in cells treated with of 9AA (1-10 μM) or doxorubicin (dox, 0.2-2 μM) for 24 h. Data are presented as relative reporter induction by the most effective dose of each chemical. F, G. Kinetics of activation of a p53-responsive reporter (F) and expression of p53-dependent targets (G) by 9AA. H. Effect of 9AA on the cell cycle in HT1080-sip53 or HT1080-siGFP cells (dox, doxorubicin, 2 μM, 24 h). J. Relative survival of the indicated isogenic pairs of tumor cells differing in p53 expression (the upper panel shows a Western analysis of p53 levels) due to transduction of lentiviral constructs expressing shRNA against p53. The lower panel shows the relative numbers of cells that survived 48 h of 9AA treatment (2 μM), compared with an untreated control. K. p53 dependence of the cytotoxicity of different drugs. The experiment described in panel J was done using 9AA (1-10 μM) doxorubicin (dox, 0.1-1 μM), camptothecin (camp, 0.16-1.6 μM), vinblastine (vinbl, 0.1-1 μM) or taxol (tax, 0.06-0.6 μM). Bars are plotted for the doses of the drugs that demonstrated the highest difference in sensitivity between p53-plus and p53-minus cells. L. 9AA is more toxic for RCC than for NKE cells. Cell numbers were estimated 72 h after treatment with the indicated concentrations of 9AA. The box shows Western analysis of the indicated proteins in lysates of NKE cells treated with doxorubicin (dox, 1 μM) or 9AA (5 μM) for 16 h.

FIG. 2. 9AA and QC activate p53 in an unusual way. A. Phosphorylation status of p53 in RCC45 cells treated with 9AA (5 μM) or doxorubicin (dox, 1 μM) for 16 h. Western analysis of total protein lysates was done by using antibodies against p53 (DO1) and against specific sites of phosphorylation in p53. B. Immunofluorescent detection of phosphorylated histone H2A.X in HT1080 cells treated with equally toxic concentrations of 9AA, QC (10 μM) or doxorubicin (1 μM). C. Western blot analysis of lysates of ACHN cells treated with 9AA (10 μM) or doxorubicin (1 μM) with antibodies against phosphorylated substrates of ATM/ATR (Cell Signaling). D. Effect of 9AA, m-AMSA and etoposide on topoisomerase II activity in vitro. E. The amount and phosphorylation status of IκB-α after treatment with 9AA. Western analyses of lysates of PC-3 cells (upper panel) treated with 9-AA (10 μM, 1-8 h) or with the proteasome inhibitor MG-132 (10 μM, 8 h) or of MCF7 cells treated with doxorubicin (dox, 1 μM), 9AA (10 μM) or cycloheximide (10 μg/ml), using antibodies specific for both phosphorylated and unphosphorylated forms of IκBα.

FIG. 3. 9AA activates p53 through inhibition of the NF-κB pathway. A. H1299-NF-kB-Luc cells were treated with different concentrations of 9AA and QC (QC) two hours before or simultaneously with TNF (10 ng/ml). Reporter activity was measured after 6 h of 9AA treatment. B. Basal activity of an NF-κB-dependent reporter was measured in lysates of H1299-NF-kBLuc cells treated with different concentrations of 9AA or QC for 24 h. C. 9AA (10 μM) inhibits reconstitution of the level of IκB protein after TNF treatment (10 ng/ml). D. sr-IκB inhibits NF-κB transcriptional activity (left panel) and activates p53 in RCC cells. Luciferase activity in ACHN cells cotransfected with p21-ConALuc and the indicated plasmids. Normalization in both assays was done by cotransfection of the pCMV-LacZ plasmid. E. Effects of NF-κB inhibition on the activity of the p53 pathway. H1299 cells were transfected with p53 alone or p53 with sr-IκB. Levels of p53 (total, using DO1 antibody, and p53 phosphorylated at serine 392), IκB, p21 and Mdm2 were analyzed by the Western method 36 h after transfection. F. The toxicity of NF-κB for tumor cells is p53-dependent. A photograph of plates with colonies of HT1080 cells—control (vector) or transduced with GSE56—after seven days in puromycin following transduction with lentiviral vectors expressing sr-IκB or insert-free vector (both conferring puromycin resistance). G. Luciferase activity in ACHN cells cotransfected with the NF-κB-responsive reporter pNF-κBLuc and sr-IκB. Twelve h after transfection, the cells were split and treated with 9AA (10 μM). NF-κB-dependent reporter activity was measured 6 h after treatment and p53-dependent reporter activity was measured 24 h after treatment. Normalization was done by cotransfection of the pCMV-LacZ plasmid (for cells transfected by different plasmids) or by determining protein concentrations (for 9AA-treated and untreated cells).

FIG. 4. Effects of 9AA on the NF-κB pathway. A. Comparison of the effects of 9AA and QC with those of known inhibitors of NF-κB. H1299-NF-κBLuc cells were treated with of 9AA (10 μM), QC (10 μM) and known inhibitors of NF-κB: sulfasalzine, capsaicin and Bay11-7082. Inhibitors of PI3K (wortmannin and Ly294002) were also assayed, since their effects on NF-κB have been demonstrated. Several effective concentrations were used, based on data in the literature, and the highest are presented. TNF (10 ng/ml) was added simultaneously (0-6 h) with inhibitors or 8 h after inhibitors (8-24 h). Luciferase activity was measured after 6 or 24 h and normalized to protein concentrations. B. 9AA causes nuclear accumulation of inactive NF-κB. H1299-NF-κBLuc cells were treated with different concentrations of 9AA and TNF (10 ng/ml). After 6 h, cytoplasmic and nuclear extracts were isolated and used for luciferase or gel-shift assays, respectively. 9AA retards the exit of p65 complexes from the nuclei. C. Immunofluorescent staining of p65 subunit of NF-κB in HT1080 cells treated with 9AA (10 μM) and TNF (10 ng/ml). D. 9AA decreases the phosphorylation of p65 in response to TNF. Upper left panel—western analysis of total cell lysates of HT1080 cells, treated with TNF (10 ng/ml) in the presence or absence of 9AA (10 μM) for the indicated periods of time. The same membrane was probed with antibodies against total p65 and against phospho-536 of p65. Lower panel—quantitation of the experiment presented in the upper left panel, using BioRad QuantityOne software. Upper right panel—western analysis with antibodies against phosphor-serine 536 of p65 in complexes immunoprecipitated from HT1080 or RCC45 cells by anti-p65 antibodies.

FIG. 5. 9AA and QC as prospective anti-RCC agents. A. Comparison of IC50 doses of 9AA, QC and several anti-cancer agents in different RCC and non-RCC cells. The IC50 for each cell line and each drug was determined as described in the Examples. Each point represents the IC50 of particular types of cells, which are grouped as follows: (i) black circles—RCC cell lines (ACHN, RCC9, RCC13, RCC29, RCC45, RCC54), (ii) red triangles—non-RCC cell lines (MCF7, HT1080, H1299, U20S, LNCaP, HCT116), (iii) green squares—normal kidney cells (NKE). B. Anti-tumor activity of QC (QC). 10⁷ ACHN cells were inoculated under the skins of nude mice. When tumors were 5 mm in diameter, intraperitoneal administration of QC was started at 50 mg/kg. 5-fluorouracil (35 mg/kg) was used as a control. Tumor size was measured every other day and is presented as the fold increase in tumor volume.

FIG. 6. 9AA treatment leads to p53 stabilization. HT1080 cells were treated or untreated with 5 μM 9AA for 16 hours. Then 9AA was substituted by 25 μg/ml of cycloheximide to block protein synthesis. Cells were lysed at indicated time points after addition of cycloheximide and p53 levels were estimated by the Western method. (A) quantitated by densitometry. Different exposure times are presented for treated and untreated cells to equalize the intensities of initial p53 signals.

FIG. 7. Comparison of survival of HT1080 cells transduced with lentiviral vectors expressing shRNA against p53 and GFP (negative control) after 72 h of treatment with the indicated concentrations of 9AA. Relative cell numbers were estimated by using a standard Methylene Blue assay.

FIG. 8. Testing the effect of CBP overexpression on p53 and NF-kB activities in HT1080 and ACHN cells. A. HT1080 cells were transfected with p53- or NF-κB-responsive reporters together with the plasmids expressing CBP, sr-IκB as a positive control and their combinations. sr-IκB and CBP plasmids were used in a broad range of concentrations to achieve saturation of their effects on the reporters. The luciferase activity was measured 48 h post-transfection. As expected, sr-IκB inhibited NF-κB-dependent transcription and activated p53. Ectopic expression of CBP increased the activity of both transcription factors in a dose-dependent manner in HT1080 cells, but the degree of p53 activation was significantly less than that caused by sr-IκB, suggesting that inhibition of NF-κB by sr-IκB leads to p53 activation through a mechanism different from the release of transcriptional co-activators. Moreover, the combination of sr-IκB and CBP had an additive effect on p53-mediated transactivation, indicating that they act through independent mechanisms. B. In the RCC-derived ACHN cell line, ectopic expression of CBP had no effect on p53-dependent transcription, although it stimulated NF-κB reporter activity. Based on these observations, we conclude that saturation of cells with CBP does not lead to effective p53 transactivation. DNA amounts in transfection mixtures were equalized using transcriptionally inert DNA

FIG. 9. 9AA causes accumulation of p65/p50 and p50/p50 NF-κB complexes in the nuclei of treated cells. Electromobility shift assay was done with nuclear extracts of H1299 cells treated for 30 minutes with 9AA (10 μM), TNF (10 ng/ml) or their combination. The specificity of the bands observed was confirmed using indicated antibodies.

FIG. 10. 9AA does not inhibit NF-κB transactivation induced by trichostatin A (TSA). H1299 cells with an integrated NF-κB-dependent luciferase reporter were treated with 100 nM of TSA in the presence or absence of 9AA (20 μM) for 4 or 16 hours. TNFα treatment was used to control for reporter activity.

FIG. 11. QC can activate p53 transactivation in organ cultures of human RCC tumor samples. Fresh surgically removed samples of tumor and adjacent normal kidney were cut into small pieces and put into media containing ConALacZ virus (5×10⁷ TU/ml) with 4 mg/ml of polybrene for 4 hours. 24 hours after infection, tissue pieces (at least three per treatment) were incubated with media containing quinacrine or doxorubicin for 12 hours. Then the tissues were fixed for 30 min in PBS containing 0.5% glutaraldehyde and 1 mM MgCl₂. Pieces of tumor were stained in PBS solution containing X-gal (1 mM MgCl₂, 3.3 mM K₄Fe(CN)₆, 3.3 mM K₃Fe(CN)₆, 0.02% NP40, 0.2% X-gal) overnight at 37° C. Stained specimens were analyzed microscopically by squeezing pieces of tumor and normal tissues between two glass slides. Treatment with QC but not doxorubicin induced expression of a p53-responsive reporter in tumor samples; no reporter induction by either drug was detectable in normal kidney samples (normal kidney structures expressing endogenous β-galactosidase activity are seen); no X-gal positive staining was observed in any tumor samples that were not transduced with the reporter.

FIG. 12. Treatment with quinacrine during antigen sensitization inhibits the contact hypersensitivity (CHS) response to antigen challenge. Quinacrine was administered to groups of 4 BALB/c mice on days −2 and −1 or on days 0 and +1. On days 0 and +1 the mice were sensitized with 0.25% DNFB by application of 25 μl to the shaved abdomen and were challenged with 0.2% DNFB on each side of both ears on day +5. The increase in ear swelling in all sensitized and challenged mice as well as in a group of naive, nonsensitized mice challenged on the ear was measured at 24 hours after the challenge and the mean is expressed in units of 10⁻⁴ in. Treatment with quinacrine just prior to sensitization decreased the CHS/ear swelling response to near the level observed following challenge of non-immune, naïve mice. In contrast, treatment with quinacrine during antigen sensitization increased the CHS response to the challenge.

FIG. 13. Treatment with quinacrine during antigen sensitization inhibits the development of antigen-specific T cells that mediate the response. Groups of mice were treated with quinacrine and sensitized with 0.25% DNFB as detailed in FIG. 12. On day +5 after antigen sensitization, CD8 T cells were prepared from the lymph nodes and cultured in triplicate with DNBS-labeled or unlabeled syngeneic spleen cells in the wells of an ELISPOT plate coated with anti-IFN-γ mAb. After 24 hours, the cells were removed and the ELISPOT assay was developed to enumerate hapten-specific CD8 T cells producing IFN-γ. The mean number of IFN-γ producing CD8 T cells in triplicate cultures is shown after subtraction of spots from control wells containing T cells with unlabeled stimulator cells (<5 spots per well). Treatment with quinacrine just prior to antigen sensitization decreased the priming of antigen-specific T cells. Similar to the induced increase in the ear swelling response, treatment with quinacrine during antigen sensitization increased the priming of the antigen-specific effector T cells almost 3-fold.

FIG. 14. Treatment with quinacrine prior to antigen sensitization results in decreased numbers of antigen-presenting dendritic cells in the skin draining lymph nodes whereas treatment during sensitization results in increased numbers of antigen-presenting dendritic cells in the lymph nodes. Quinacrine was administered to BALB/c mice on days −2 and −1 or on days 0 and +1. The quinacrine treated and non-treated mice were sensitized with 1% FITC on days 0 and +1 and on day +2, skin draining lymph node cell suspensions were stained for the dendritic cell marker CD11c and the presence of CD11c+/FITC+ cells in the lymph nodes was analyzed by flow cyotmetry. The decrease and increase in FITC+ dendritic cells correlates with the numbers of hapten-specific T cells producing IFN-γ shown in FIG. 13.

FIG. 15. Treatment with quinacrine prior to antigen sensitization decreases antigen-induced mRNA expression and protein production of proinflammatory cytokines in the skin. Quinacrine was administered to BALB/c mice on days −2 plus −1. On day 0 the mice were sensitized with 0.25% DNFB by application of 25 μl to the shaved abdomen and 3 hours later the sensitized skin was excised and total RNA was extracted. A. The expression of IκBα, IL-1β, and TNFα was assessed using semi-quantitative RT-PCR (reverse transcriptase-polymerase chain reaction). B. Densitometric analysis was performed to quantify cytokine mRNA signals in comparison to the GAPDH signal. C. The expression of CCL21/SLC was tested in the skin mRNA. D. Total protein extracts were prepared from the skin of naïve or sensitized control and quinacrine-treated mice at 8, 20, and 30 hours after sensitization and tested for IL-1β and TNFα production by ELISA. The results are expressed as mean concentration of cytokine per 5 mg of total protein ±SEM (standard error of the mean).

FIG. 16. Treatment with quinacrine during immunization increases antibody production. Groups of 3 BALB/c mice were immunized intraperitoneally with 100 ug of ovalbumin on days 0 and 7 with or without treatment with quinacrine (80 mg/kg) on days 1, 2, 8 and 9. On day 14 serum was obtained from all immunized mice and from two unimmunized (naïve) mice. Serial dilutions of each serum sample were placed in wells of 96 well plates coated with or without ovalbumin and the ovalbumin-specific IgG titers of each animal were determined by ELISA. Treatment with quinacrine increased the antigen-specific titers of IgG 4-fold.

FIG. 17. Groups of 5 C57BL/6 mice were treated with quinacrine on days −2, −1, +5, +14, and +16 and received heterotopically transplanted complete MHC-mismatched heart grafts from A/J donors on day 0. Control recipients received PBS without quinacrine. The survival of the heart allografts was monitored daily by abdominal palpation and rejection was confirmed by laparotomy. Quinacrine treatment extended the survival of all grafts with the absence of rejection in one of the allografts.

### DETAILED DESCRIPTION

NF-κB is the major regulator of innate and adaptive immune responses, including activation of cytokine and chemokine release, induction of proliferation of immune cells, establishment of resistance to apoptotic stimuli, etc. Inhibition of NF-κB can result in abrogation of the above mentioned processes that under certain circumstances may be involved in disease conditions such as acute inflammation (i.e., septic shock), chronic inflammation, fibrotic diseases, tumor resistance to treatment, autoimmune diseases, rejection of allograft transplants, graft versus host disease, allergic reactions, etc. Inhibitors of IKK2, the kinase positioned upstream of NF-κB in the pathway and involved in NF-κB regulation, are used or being considered for treatment of the above conditions. As demonstrated in these Examples, quinacrine and related compounds with similar mechanism of activity form a previously unknown type of NF-κB inhibitor that may not trap NF-kB in the cytoplasm, as do IKK2 inhibitors, but may convert NF-κB complex from transactivator into a transrepressor causing inhibition of NF-κB function. As a result, these compounds may be used to module the immune responses for treatment a series of pathologies involving unwanted immune reactions. The compounds may also be used to treat allergies and transplant recipients may provide significant advantages over existing therapies and protocols provided by the high degree of safety of compounds such as quinacrine in humans that has no record of adverse effects that would provoke the occurrence of end-stage renal dysfunction or autoimmune or infectious diseases.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

The term “branched” as used herein refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, —CH₂CH₂CH(CH3)CH₂CH₃, —CH₂CH(CH₂CH₃)CH₂CH₃, —CH₂CH₂C(CH₃)₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group having 4n+2 delocalized π(pi) electrons, which may be substituted or unsubstituted. The term aromatic further includes aromatic groups, which comprise a nitrogen atom replacing one or more carbons of the hydrocarbon backbone. Examples of aromatic groups include, but are not limited to, phenyl, naphthyl, thienyl, furanyl, pyridinyl, (is)oxazoyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di,” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, combinations thereof, and substitutions thereto.

The term “unsubstituted” as used herein refers to a group that does not have any further groups attached thereto or substituted therefor.

The term “alkyl” as used herein alone or in combination refers to a branched or unbranched, saturated aliphatic group. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like.

The term “alkenyl” as used herein alone or in combination refers to a branched or unbranched, unsaturated aliphatic group containing at least one carbon-carbon double bond which may occur at any stable point along the chain. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. Representative examples of alkynyl groups include, but are not limited to, ethynyl, propynyl, propargyl, butyryl, hexynyl, decynyl and the like.

The term “aryl” as used herein alone or in combination refers to a substituted or unsubstituted aromatic group, which may be optionally fused to other aromatic or non-aromatic cyclic groups. Representative examples of aryl groups include, but are not limited to, phenyl, benzyl, naphthyl, benzylidine, xylyl, styrene, styryl, phenethyl, phenylene, benzenetriyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The term “halogen,” “halide” or “halo” as used herein alone or in combination refers to fluorine “F”, chlorine “Cl”, bromine “Br”, iodine “I”, and astatine “At”. Representative examples of halo groups include, but are not limited to, chloroacetamido, bromoacetamido, idoacetamido and the like.

The term “hetero” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” as used herein refers to a cyclic group containing a heteroatom. Representative examples of heterocycles include, but are not limited to, pyridine, piperadine, pyrimidine, pyridazine, piperazine, pyrrole, pyrrolidinone, pyrrolidine, morpholine, thiomorpholine, indole, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “acryl” as used herein alone or in combination refers to a group represented by CH₂═C(Q)C(O)O— where Q is an aliphatic or aromatic group.

The term “cyano,” “cyanate,” or “cyanide” as used herein alone or in combination refers to a carbon-nitrogren double bond. Representative examples of cyano groups include, but are not limited to, isocyanate, isothiocyanate and the like.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thiol, thioxos and the like.

The term “optional” or “optionally” as used herein means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both unsubstituted alkyl and alkyl where there is a substitution.

The term “effective amount,” when used in reference to a compound, product, or composition as provided herein, means a sufficient amount of the compound, product or composition to provide the desired result. The exact amount required will vary depending on the particular compound, product or composition used, its mode of administration and the like. Thus, it is not always possible to specify an exact “effective amount.” However, an appropriate effective amount may be determined by one of ordinary skill in the art informed by the instant disclosure using only routine experimentation.

The term “suitable” as used herein refers to a group that is compatible with the compounds, products, or compositions as provided herein for the stated purpose. Suitability for the stated purpose may be determined by one of ordinary skill in the art using only routine experimentation.

As used herein, the terms “administer” when used to describe the dosage of a compound, means a single dose or multiple doses of the compound.

As used herein, “apoptosis” refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer” means any condition characterized by resistance to apoptotic stimuli.

As used herein, the term “cancer treatment” means any treatment for cancer known in the art including, but not limited to, chemotherapy and radiation therapy.

As used herein, the term “combination with” when used to describe administration of an aminoacridine and an additional treatment means that the aminoacridine may be administered prior to, together with, or after the additional treatment, or a combination thereof.

As used herein, the term “treat” or “treating” when referring to protection of a mammal from a condition, means preventing, suppressing, repressing, or eliminating the condition. Preventing the condition involves treating the mammal prior to onset of the condition. Suppressing the condition involves treating the mammal after induction of the condition but before its clinical appearance. Repressing the condition involves treating the mammal after clinical appearance of the condition such that the condition is reduced or maintained. Elimination the condition involves treating the mammal after clinical appearance of the condition such that the mammal no longer suffers the condition.

As used herein, the term “tumor cell” means any cell characterized by resistance to apoptotic stimuli.

As used herein, the term “mammal” includes humans, companion animals (e.g., dogs, cats and horses), zoo animals (e.g., zebras, elephants, etc.), food-source animals (e.g., cows, pigs, goats, and sheep) and research animals (e.g., rats, mice, goats, guinea pigs, etc.).

### 2. MODULATION OF NF-κB-MEDIATED IMMUNE RESPONSE

The present invention is related to the discovery that aminoacridines modulate the effect of NF-κB-mediated immune responses. Aminoacridines may be administered to inhibit NF-κB-mediated immune responses. Aminoacridines may be administered to stimulate NF-κB-mediated immune responses.

### 3. AMINOACRIDINES

Aminoacridines are representative examples of agents which may be used to modulate NF-κB-mediated immune responses. The aminoacridine may be of the following formula:

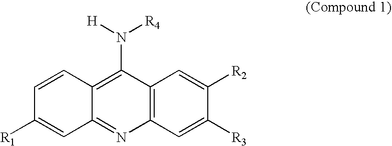

wherein,
R₁ is H or halogen;
R₂ is H or optionally substituted alkoxy;
R₃ is H or optionally substituted alkoxy; and
R₄ is H or optionally substituted aliphatic, aryl, or heterocycle.

Representative examples of aminoacridines include, but are not limited to, 9-aminoacridine or Mepacrine, which is otherwise known as Quinacrine, as well as those aminoacridines described in PCT/US05/25884, the contents of which are incorporated herein by reference. The use of aminoacridines to modulate NF-κB-mediated immune responses is attractive because many aminoacridines have limited side effects.

### 4. COMPOSITIONS

The present invention relates to a composition comprising an aminoacridine and optionally a chemotherapeutic.

a. Formulations

The composition may further comprise one or more pharmaceutically acceptable additional ingredient(s) such as alum, stabilizers, antimicrobial agents, buffers, coloring agents, flavoring agents, adjuvants, and the like.

The composition may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

The composition may also be liquid formulations including, but not limited to, aqueous or oily suspensions, solutions, emulsions, syrups, and elixirs. The composition may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The composition may also be formulated as suppositories, which may contain suppository bases including, but not limited to, cocoa butter or glycerides. The composition may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. The composition may also be formulated transdermal formulations comprising aqueous or nonaqueous vehicles including, but not limited to, creams, ointments, lotions, pastes, medicated plaster, patch, or membrane.

The composition may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

The composition may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes may be used such as those described in U.S. Pat. No. 5,077,211, U.S. Pat. No. 4,621,023 or U.S. Pat. No. 4,508,703, which are incorporated herein by reference. A composition intended to target skin conditions can be administered before, during, or after exposure of the skin of the mammal to UV or agents causing oxidative damage. Other suitable formulations can employ niosomes. Niosomes are lipid vesicles similar to liposomes, with membranes consisting largely of non-ionic lipids, some forms of which are effective for transporting compounds across the stratum corneum.

### 5. TREATMENT

The composition may be used for treating a condition associated with NF-κB-mediated immune response in vivo by administering to a patient in need thereof an aminoacridine. The composition may be used to inhibit NF-κB-mediated immune responses. The composition may also be used to increase NF-κB-mediated immune responses, for examples as an adjuvant therapy

a. Administration

The composition may be administered simultaneously or metronomically with other treatments. The term “simultaneous” or “simultaneously” as used herein, means that the other treatment and the composition is administered within 48 hours, 24 hours, 12 hours, 6 hours, 3 hours or less, of each other. The term “metronomically” as used herein means the administration of the composition at times different from the chemotherapy and at certain frequency relative to repeat administration and/or the chemotherapy regiment.

The composition may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. The composition may also be administered in the form of an implant, which allows slow release of the composition as well as a slow controlled i.v. infusion.

b. Dosage

A therapeutically effective amount of an agent required for use in therapy varies with the nature of the condition being treated, the length of time that activity is desired, and the age and the condition of the patient, and is ultimately determined by the attendant physician. The desired dose may be conveniently administered in a single dose, or as multiple doses administered at appropriate intervals, for example as one, two, three, four or more subdoses per day. Multiple doses often are desired, or required.

When given in combination with other therapeutics, the composition may be given at relatively lower dosages. In addition, the use of targeting agents may allow the necessary dosage to be relatively low. Certain compositions may be administered at relatively high dosages due to factors including, but not limited to, low toxicity, high clearance, low rates of cleavage of the tertiary amine. As a result, the dosage of a composition may be from about 1 ng/kg to about 200 mg/kg, about 1 μg/kg to about 100 mg/kg, or about 1 mg/kg to about 50 mg/kg. The dosage of a composition may be at any dosage including, but not limited to, about 1 mg/kg, 25 μg/kg, 50 μg/kg, 75 μg/kg, 100 μg/kg, 125 mg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μm/kg, 475 μg/kg, 500 μg/kg, 525 μg/kg, 550 μg/kg, 575 μg/kg, 600 μg/kg, 625 μg/kg, 650 μg/kg, 675 mg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

### 6. SCREENING METHODS

The present invention also relates to methods of identifying agents that modulate NF-κB mediated immune responses. An agent that modulates NF-κB activity may be identified by a method comprising adding a candidate modulator of NF-κB activity to a cell-based NF-κB activated expression system, whereby a modulator of NF-κB activity is identified by the ability to alter the level of NF-κB activated expression. An agent that modulates NF-κB activity may also be identified by a method comprising adding a candidate modulator of NF-κB activity to a cell-based p53 activated expression system, whereby a modulator of NF-κB activity is identified by the ability to alter the level of p53 activated expression. An agent that modulates NF-κB activity may also be identified by a method comprising adding an aminoacridine and a candidate modulator of NF-κB activity to an NF-κB or p53 activated expression system, comparing the level of NF-κB or p53 activated expression to a control, whereby a modulator of NF-κB activity is identified by the ability to alter the level of NF-κB or p53 activated expression system compared to the control.

The cell may comprise a functionally silent p53. The cell may also comprise an NF-κB transactivation complex. The p53 activated expression system may be in a renal carcinoma cell line. The cell line may also be a sarcoma cell line. The cell line may also be a cell line with amplified mdm2. The cell line may also be a cell line that expresses HPV-E6 or is capable thereof.

Candidate agents may be present within a library (i.e., a collection of compounds). Such agents may, for example, be encoded by DNA molecules within an expression library. Candidate agent be present in conditioned media or in cell extracts. Other such agents include compounds known in the art as “small molecules,” which have molecular weights less than 10⁵ daltons, preferably less than 10⁴ daltons and still more preferably less than 10³ daltons. Such candidate agents may be provided as members of a combinatorial library, which includes synthetic agents (e.g., peptides) prepared according to multiple predetermined chemical reactions. Those having ordinary skill in the art will appreciate that a diverse assortment of such libraries may be prepared according to established procedures, and members of a library of candidate agents can be simultaneously or sequentially screened as described herein.

The screening methods may be performed in a variety of formats, including in vitro, cell-based and in vivo assays. Any cells may be used with cell-based assays. Preferably, cells for use with the present invention include mammalian cells, more preferably human and non-human primate cells. Cell-base screening may be performed using genetically modified tumor cells expressing surrogate markers for activation of NF-κB and/or p53. Such markers include, but are not limited to, bacterial β-galactosidase, luciferase and enhanced green fluorescent protein (EGFP). The amount of expression of the surrogate marker may be measured using techniques standard in the art including, but not limited to, colorimetery, luminometery and fluorimetery. Representative examples of cells that may be used in cell-based assays include, but are not limited to, renal cell carcinoma cells.

The conditions under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and pad plates. Culturing is carried out at a temperature, pH and carbon dioxide content appropriate for the cell. Such culturing conditions are also within the skill in the art.

Methods for adding a suspected modulator to the cell include, but are not limited to, electroporation, microinjection, cellular expression (i.e., using an expression system including naked nucleic acid molecules, recombinant virus, retrovirus expression vectors and adenovirus expression), use of ion pairing agents and use of detergents for cell permeabilization.

The present invention has multiple aspects, illustrated by the following non-limiting examples.

### EXAMPLES

### Materials and Methods

### 1. Cells

The renal cell carcinoma cell lines used, RCC45, RCC54 and ACHN were previously described (1a), RCC9, RCC13, RCC28 and RCC29 cells were provided by Dr. J. Finke (Cleveland Clinic Foundation). H1299, HT1080, MCF7, LNCaP, HCT116, U2OS, WI38 cells were obtained from ATCC. All cells were maintained in RPMI 1640 medium, supplemented with 10% FBS, 1 mM sodium pyruvate, 10 mM Hepes buffer, 55 nM β-mercaptoethanol and antibiotics. Reporter cell lines with Myc, or Clock/Bmal responsive reporters were kindly provided by C. Burkhart and M. Antoch (Cleveland Clinic Foundation, OH). Cells with inhibited p53 expression were generated by retroviral transduction of pBabeH1-sip53 or pBabeH1-siGFP vectors for siRNA expression with following selection on puromycin.

### 2. Plasmids

p53 and Arf expression vectors and the pBabeH1-siHdm2 and p21-ConALuc reporter plasmids are described in (1a). The pNF-κBLuc plasmid was provided by N. Neznanov (Cleveland Clinic Foundation, ref. 60). The pcDNA3 vector expressing pss-IκB was provided by Inder Verma (Salk Institute for Biological Sciences, CA). The pCBP plasmid was provided by Tony Kong (Rutgers University, NJ). The pBabeH1-sip53 and pBabeH1-siGFP vectors for siRNA expression were generated by insertion of the H1promoter and a 64 oligonucleotide loop template for siRNA expression (2a) into the left LTR of the pBabeH1-puro vector analogously to the pBabeH1-siHDM2 vector, described in (1a). Sequences for siRNAs against p53 and GFP have been described (2a). Lentiviral plasmids for p53 or GFP expression have been described (1a). Lentiviral vectors carrying p53-responsive β-galactosidase (LacZ) and NF-κB-responsive GFP were provided by Peter Chumakov (Lerner Research Institute Cleveland Clinic Foundation), lentiviral vectors with p53-responsive reporters were made by substitution of the CMV promoter in the pLV-CMV-Luc plasmid in place of the ConA element.

### 3. Retroviral and Lentiviral Transductions

Stocks of recombinant lentiviruses carrying p53, EGFP (control vector) or p53-responsive LacZ and NF-κB-responsive EGFP were prepared using the 293 cell line transfected with pLV-CMV-p53, pLV-CMV-EGFP, pLV-ConALacZ or pLV-NF-κB-EGFP plasmids along with packaging plasmids encoding viral structural proteins and the G-protein of vesicular stomatitis virus (provided by Inder Verma, Salk Institute for Biological Sciences, CA), using lipofectamine reagent (Invitrogen). Virus-containing media from 293T cells were collected 48 hours later and virus was concentrated 50-100 times by ultracentrifugation. Virus titers (typically 10⁸ TU/ml) were determined by immunofluorescent staining for p53, GFP fluorescence or X-gal staining.

### 4. Reporter Assays

Transient transfections. 2×10⁵ cells were plated into 6-well plates and, after overnight incubation, transfected with Lipofectamin Plus reagent (Gibco BRL) with 0.5 μg of reporter plasmids (p21-ConALuc or pNF-κBLuc) in combination with different concentrations of plasmids expressing p53, Arf, Ss-IκB, or siHDM2. Corresponding empty vectors were added in all transfections up to 2 μg of total DNA. Normalization of transfection efficiency was done by adding 0.2 μg of pCMV-LacZ plasmid. Luciferase activity and β-galactosidase activity were measured in lysates prepared 48 hours after transfection with Cell Lysis Buffer (Promega) by the luciferase assay system (Promega) or β-galactosidase enzyme system (Promega). Luminometric and colorimetric reactions were read on the Wallack 1420 plate reader (Perkin Elmer). Stable transduction of reporters. 2×10⁴ cells with integrated reporter were plated in 96-well plates. After overnight incubation, chemicals or media from lentivirus-producing cells were added. At different times, cell lysates were prepared using Reporter Lysis Buffer (Promega). Luciferase or β-galactosidase activity and protein concentration were measured in aliquots of cell lysates using standard kits (Promega, Luciferase and β-galactosidase assay systems, Biorad Protein Assay Kit).

5. Screening for p53-Activating Compounds

The DiverSet library of 34,000 chemical compounds and focused libraries of structural analogues of selected hits were provided by Chembridge, Calif. Cell lines with p53- or NF-κB-responsive luciferase or β-galactosidase reporters were described previously (5, 7). For screening chemicals, 2×10⁴ RCC45ConALacZ cells (RCC45-based p53 reporter cells) were plated per well of 96-well plates in 200 μL of phenol-red-free RPMI medium with standard additions. After overnight incubation, the compounds were added to final concentrations of 5-15 μM (0.2 μL of compound in DMSO per well). Equal amounts of DMSO and doxorubicin (0.2-2 μM) were used as controls. After 24 h, lysis buffer with ortho-nitrophenyl-β-D-galactopyranoside (ONPG) was added and β-galactosidase activity was estimated colorimetrically by a multiwell plate reader. Compounds that induced the reporter stronger than doxorubicin (the only drug tested that was capable of weakly activating the p53-dependent reporter in RCC), were considered to be primary hits.

### 6. Cell Survival Assays

Clonogenic survival. 5×10³ cells were plated in 6-well plates and treated with different concentrations of drugs for 24 hours. Then fresh drug-free medium was added and the numbers of colonies were estimated 7-14 days later. Growth inhibition assay. 5×10³ cells were plated per well of 96-well plates and the next day different concentrations of 9AA, QC, doxorubicin, Taxol or 5-fluorouracil were added for 24 hours. Cell survival was estimated after an additional 48 hours, using standard Methylene Blue staining followed by extraction and colorimetrical detection of the dye.

### 7. Cell Cycle Analysis

10⁶ cells were plated in 100-mm plates and, after overnight incubation, different concentrations of 9AA or doxorubicin were added. At the end of the incubation period, cells were collected, fixed and stained with propidium iodide as previously described (3a). DNA content was measured using FACScalibur (Becton Dickinson) and analyzed using CellQuest software.

### 8. Western Analysis

Cells were lysed in RIPA buffer (25 mM Tris HCl, pH 7.2, 125 mM NaCl, 1% NP40, 1% sodium deoxycholate, 1 mM EDTA) containing 1 mM PMSF (Sigma), 10 ug/ml of aprotinin (Sigma) and 10 ug/ml of leupeptin (Sigma). Protein concentrations were determined with the BioRad Dc protein assay kit. Equal protein amounts were run on gradient 4-20% precast gels (Novex) and blotted onto PVDF membranes (Amersham). The following antibodies were used: anti-p53—monoclonal mouse DO1 (Santa-Cruz), anti-p21—monoclonal mouse F-5 (Santa-Cruz), anti-mdm2—monoclonal mouse SMP14 (Santa-Cruz), anti-p65—(C20, Santa Cruz), anti-phospho-p65—(ser536, Cell Signaling), anti-IκBa—(C21, Santa Cruz), anti-p50—(NLS, Santa Cruz), anti-IKK1 (Cell Signaling), anti-γH2X (Cell Signaling), anti-actin (Santa Cruz). p53 phosphorylation status was analyzed using the phospho-p53 sampler kit from Cell Signaling according to the manufacturer's recommendations. HRP-conjugated secondary antibodies were purchased from Santa Cruz. Quantitation of the data was performed using Quantity One software from BioRad.

### 9. Immunofluorescent Staining

Cells on chamber slides were washed with PBS and fixed subsequently with 10% phosphate-buffered formalin at room temperature, 100% methanol at −20° C. and acetone at −20°. Then slides were blocked in a solution of 3% BSA, 0.1% Triton X100 in PBS for 1 hour. Primary antibodies were added at a concentration of 1 μg/ml in blocking solution. Secondary anti-rabbit Cy2-conjugated antibodies (Sigma) were used. All washings were done with blocking solution. Staining of phosphorylated histone 2AX was done according to the manufacturer's protocol.

### 10. DNA Topoisomerase II Activity Assay

HT1080 cells were labeled for 24 hours with 0.02 to 0.04 mCi/mL of [¹⁴C]thymidine, specific activity 53 mCi/mmol (Amersham). The labeled HT1080 cells were treated with different concentrations of etoposide (VP-16), m-AMSA, or 9-aminoacridine for 1 h. The induction of topo II-mediated DNA scission was determined by measuring precipitation of the protein DNA complex using a modification of the SDS-KCl technique (4a-6a).

### 11. Proteasome Inhibitor Assay

The kit for assaying proteasomal activity was purchased from Boston Biochem, Inc. and used according to the manufacturer's recommendations.

### 12. Electromobility Shift Assay (EMSA)

Nuclear extracts were prepared as already described (7a). Annealed oligonucleotide, corresponding to an NF-κB binding site (Santa-Cruz), was radio-labeled with [alpha-³²P]dCTP by using the Klenow polymerase and then with [gamma-³²P]dATP by using T4 polynucleotide kinase. 10⁷ cpm of labeled oligonucleotide was affinity purified on Probe Quant columns (Amersham). Radio-labeled oligonucleotide were added to 10 μg of protein nuclear extract together with 1 μg of poly-dIdC (Amersham) to prevent nonspecific binding and incubated for 30 min at room temperature. For supershift assays, 200 ng of anti-p65, anti-p50 or anti-antibodies were added to the reaction (all antibodies are from Santa Cruz). After 30 min incubation, entire reaction mixtures were loaded into 4% polyacrilamide gel in 0.5×TBE buffer and run for 2 hours at 200V. Dried gels were exposed to X-ray films for 30-60 min.

### 13. Experimental Chemotherapy of Tumor Xenografts

NIH Swiss athymic nude, male mice, 5-6 weeks old, were purchased from Harlan. 5×10⁶ tumor cells were inoculated into the flanks of mice in 100 μL of PBS. When tumors reached 5 mm in diameter, intraperitoneal injections of drugs were started in 100 μL of a solution of 50% DMSO in PBS (except for quinacrine, which was dissolved in PBS). As vehicle, 50% DMSO solution in PBS was used. Tumor size was measured in three dimensions every other day.

14. Gene Expression Profiling of RCC Cells Treated with 9AA

We analyzed by microarray hybridization changes in global gene expression profiles in RCC45 and RCC54 cells treated with 2 μM or 10 μM of 9AA, which cause growth arrest or apoptosis, respectively. RNA was isolated after 16 hours of treatment, enough to induce p53 but before the appearance of signs of toxicity. Among the 36,847 human genes present on the NimbleGen oligonucleotide array, only 0.6% changed expression two-fold or more with one dose of 9AA in both cell lines. p21, Mdm2, as well as several other p53 targets were among the most genes most highly upregulated (Table 1), while genes encoding IκBα, IL-8 and several chemokines and cytokines, all known to be NF-κB-responsive, were strongly suppressed as a result of treatment (Table 2).

### 15. Hapten Sensitization and Elicitation of Contact Hypersensitivity

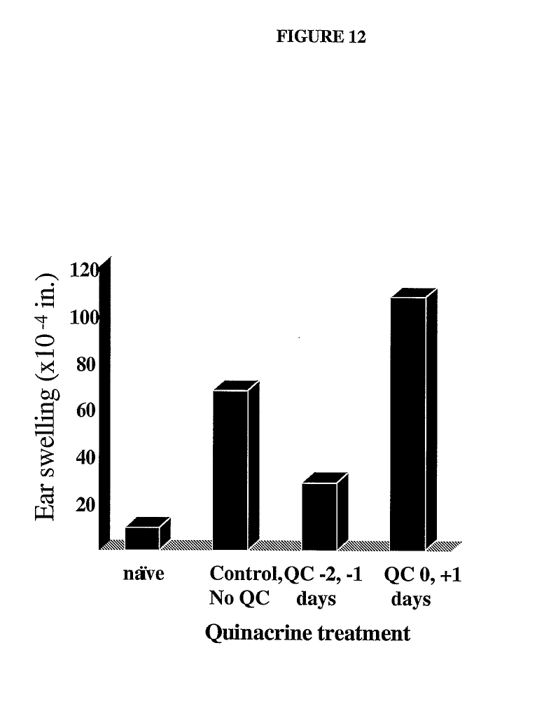

For sensitization to DNFB, mice were painted on days 0 and +1 with 25 ml of 0.25% 2,4-dinitrofluorobenzene (DNFB) (Sigma Chemical, Co.) on the shaved abdomen and 5 ml on each footpad. On day +5 sensitized and, as a negative control, unsensitized mice were challenged with 10 ml of 0.2% DNFB on both sides of each ear. Ear thickness was measured in a blinded manner at 24 h intervals after challenge using an engineer's micrometer (Mitutoyo, Elk Grove Village, Ill.) and expressed in units of 10⁻⁴ inches. The magnitude of ear swelling responses is presented in FIG. 12 as the mean increase of each group of 4 sensitized or non-sensitized mice (i.e. 6 ears) ±SEM over the thickness measured just prior to hapten challenge on day +5 post-sensitization. To test the effect of quinacrine (QC) on CHS responses, groups of mice were injected i.p. with 80 mg/kg QC diluted in sterile PBS on days −2 and −1 before hapten sensitization. Control mice received injections with an equal volume of PBS.

### 16. ELISPOT Assays

ELISPOT assays to enumerate hapten-specific T cells producing IFN-γ were performed as previously described [21]. Briefly, ELISPOT plates (Millipore, Bedford, Mass.) were coated with 100 ml of 4 mg/ml anti-IFN-γ mAb R26A2 and incubated overnight at 40° C. The plates were blocked with 1% BSA in PBS for 90 min at 37° C. and washed four times with PBS. LNC suspensions from DNFB-sensitized mice were prepared on day +5 after sensitization and CD8 + T cells were enriched by negative selection using Dynabeads (Dynal A. S., Oslo, Norway). The efficiency of CD4+ T cell depletion was >95% as assessed by flow cytometry. Syngeneic spleen cells from naive mice were treated with 50 mg/ml mitomycin C, hapten-labeled by incubation with DNBS (100 mg/ml), and used as stimulator cells. Responder LNC were resuspended in serum-free HL-1 medium (BioWhittaker, Walkersville, Md.) and cultured at 3×10⁵ cells/well with 5×10⁵ stimulator cells/well 24 h at 37° C. in 5% CO₂. In all experiments responder CD8+ T cells cultured with unlabeled splenocytes and LNC from naive mice cultured with DNBS-labeled stimulator cells were used as negative controls. After 24 h, cells were removed from the culture wells by extensive washing with PBS and then PBS/0.2% Tween 20 (PBS-T). Biotinylated anti-IFN-γ mAb XMG1.2 (2 mg/ml) was added and the plate was incubated overnight at 40° C. The following day the wells were washed three times with PBS-T and incubated with anti-biotin mAb conjugated with alkaline phosphatase for IFN-γ. After 2 h at RT the wells were washed with PBS and nitroblue tetrazolium 5-bromo-4-chloro-3-indolyl substrate (Kirkegaard & Perry Laboratories, Gaithersburg, Md.) was added for the detection of IFN-γ. The resulting spots were counted on an ImmunoSpot Series 1 Analyzer (Cellular Technologies Ltd., Cleveland, Ohio) that was designed to detect spots with predetermined criteria for size, shape and colorimetric density. To determine the number of hapten-specific cytokine-producing T cells, the number of spots resulting from the culture of T cells with unlabeled splenocytes (typically less than 5 spots per well) was subtracted from the number of spots resulting from the culture of T cells with hapten-labeled cells.

### 17. Flow Cytometry

LNC were obtained from hapten-sensitized mice on day +2 post-sensitization. To prevent non-specific antibody binding cells were incubated with rat serum (Rockland, Gilbertsville, Pa.) diluted 1:1000 in staining buffer (Dulbecco's PBS with 2% FCS/0.02% NaN₃) for 20 min on ice. Then cells were washed and stained with PE-labeled anti-CD11c mAb to detect DC. Stained cells were washed five times, resuspended in staining buffer and analyzed by two-color flow cytometry using a FACScan (Becton Dickinson, San Jose, Calif.).

To detect Langerhans cells in the lymph nodes LNC were obtained 48 h after sensitization with 1% FITC and were stained with PE-labeled anti-CD11c mAb and then analyzed by two-color flow cytometry.

### 18. Analysis of IL-1B, TNF-α, CCL21 and IκBα Expression by RT-PCR

Naive and sensitized mouse skin (˜200 mg) was snap frozen in liquid nitrogen and then ground using a mortar and pestle in liquid nitrogen. Total RNA was extracted using the RNeasy Tissue Kit (Qiagen, Inc., Valencia, Calif.) according to the manufacturer's protocol. For synthesis of cDNA, 2 mg of total RNA from each sample was resuspended in 10 ml of the reaction buffer. cDNA synthesis was performed using M-MLV reverse transcriptase (Promega Corporation, Madison, Wis.) with random primers according to the manufacturer's protocol. The reaction was stopped by denaturing the enzyme at 99° C. for 5 min. and the mixture was diluted with distilled water to a final volume of 50 ml. Aliquots (3 ml) of the synthesized cDNA were added to 47 ml of PCR mixture, containing 1×PCR buffer, MgCl₂ (1.5 mM), dNTP (0.2 mM each), primer mix (0.5 mM each) and 2.0 units of Taq DNA polymerase (all from Invitrogen Corp., Carlsbad, Calif.). PCR primers for mouse GAPDH, IκBα, TNF-α, IL-1β and CCL21 were purchased from Integrated DNA Technologies, Inc. (Coralville, Iowa) and contained the following sequences:

| | |
|---|---|
| | SEQ |
| | ID |
| SEQUENCE | NO |

mGAPDH

| | | |
|---|---|---|
| sense | 5′-TCGTGGATCTGACGTGCCGCCTG-3′ | 1 |
| anti-sense | 5′-CACCACCCTGTTGCTGTAGCCGTAT-3′ | 2 |
| mIκBα | | |
| sense | 5′-CTGGGAGCTGGCTGTGATCC-3′ | 3 |
| anti-sense | 5′-TCTGTGTCATAGCTCTCCTCA-3′ | 4 |
| mIL-1β | | |
| sense | 5′-AAATGCCTCGTGCTGTCTGACC-3′ | 5 |
| anti-sense | 5′-CTGCTTGAGAGGTGCTGATGTACC-3′ | 6 |
| mTNF-α | | |
| sense | 5′-ATGAGCACAGAAAGCATGATCCGC-3′ | 7 |
| anti-sense | 5′-CCAAAGTAGACCTGCCCGGACTC-3′ | 8 |
| mCCL21 | | |
| sense | 5′-CAGGACTGCTGCCTTAAGTA-3′ | 9 |
| anti-sense | 5′-GCACATAGCTCAGGCTTAGA-3′ | 10 |

Amplification was initiated by 1 min of denaturation at 95° C. for 1 cycle. This was followed by 25-35 cycles at 94° C. for 45 sec, 56° C. for 30 sec, and 72° C. for 30 sec. After the last amplification cycle, the samples were incubated for 7 min at 72° C. For each set of primers, dilutions of cDNA were amplified for 20-40 cycles to define optimal conditions for linearity and to permit semi-quantitative analysis of signal strength. Abundant mRNAs (i.e. GAPDH and IκBα) were amplified 25 cycles whereas cytokine mRNAs were amplified 30-35 cycles.

The amplified PCR products were separated on a 2% agarose gel with 1× tris-acetate buffer at 75V for 40-60 min and visualized by UV light after staining with 0.5 mg/ml ethidium bromide. Gels were photographed using the Bio Doc-It Imaging System (UVP, Inc., Upland, Calif.). Densitometric analysis of the captured images was performed using NIH 1.54 image analysis software and normalized to the GAPDH content.

### 19. Measurement of TNF-α and IL-β Proteins in the Skin

To test TNF-α and IL-β production in sensitized skin, skin was excised at indicated times after DNFB sensitization and homogenized in the presence of proteinase inhibitors (10 mg/ml PMSF, 2 mg/ml aprotinin, 2 mg/ml leupeptin and 100 mg/ml chymostatin). Following standardization of protein levels to 5 mg total protein/ml, aliquots were tested by ELISA to quantitate TNF-α and IL-1β levels using Quantikine Mouse Cytokine Immunoassay Kits (R & D Systems Inc., Minneapolis, Minn.).

### 20. Heterotopic Cardiac Transplantation.

Heterotopic cardiac transplants were performed using microsurgical techniques. Briefly, donor hearts were harvested and placed in chilled Ringer's solution while the recipient mice were prepared. The donor heart was anastomosed to the recipient abdominal aorta and inferior vena cava using microsurgical techniques. Upon completion of the anastomoses and organ reperfusion, the heart grafts resumed spontaneous contraction. The strength and quality of cardiac graft impulses were monitored each day by palpation of the abdomen. Rejection of cardiac grafts was considered complete by cessation of impulse and was confirmed visually for each graft by laparotomy. Isografts were maintained in syngeneic recipients beyond 100 days.

### Example 1

### 9AA and QC Activate p53 Through an Unusual Mechanism

p53 controls genetic stability and reduces the risk of cancer through induction of growth arrest or apoptosis in response to DNA damage or deregulation of proto-oncogenes (1). The efficacy of p53 as a tumor-preventing factor is reflected by the high frequency of p53 loss, in at least 50% of human tumors, due to inactivating mutations (2). Understanding the mechanisms of functional inactivation of wild type p53 in human tumors, for example, by overexpression of natural antagonists of p53, Mdm2 or the viral protein E6, helps to define prospective targets for treating cancer by restoring p53 function (3).

We have recently shown that renal cell carcinomas, the most frequent and least curable type of kidney cancer, maintain wild-type but functionally inactive p53 (4). The mechanism of p53 repression in RCC is dominant, and therefore “druggable”, and different from that of all reported cases of p53 repression in tumors, suggesting the existence of an as yet unknown molecular target for restoring p53 function in cancer. As an approach to finding such factor(s), we set out to isolate compounds that are capable of restoring p53 function in RCC and strongly activate p53 in many other types of cancer cells.

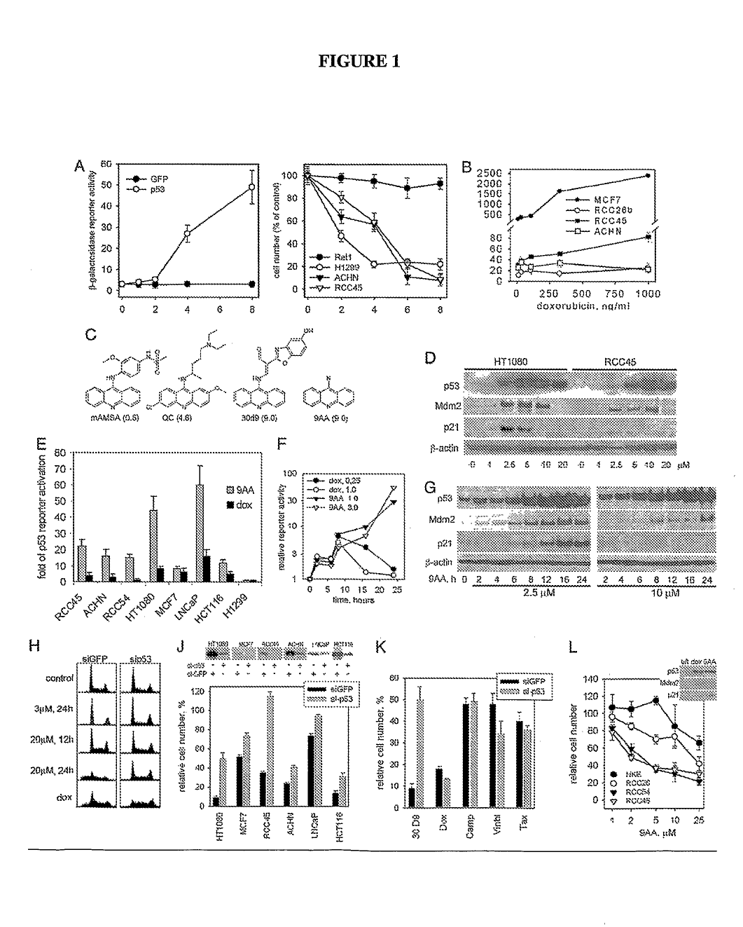

To test whether p53 reactivation in RCC is achievable in principle, we expressed increasingly high levels of wild type p53 in RCC-derived cell lines carrying an integrated p53-responsive reporter (5) using lentiviral transduction. At a certain level of expression, p53 became simultaneously cytotoxic and capable of inducing the reporter (FIG. 1A), presumably by depleting a factor that inhibits p53, thus justifying the isolation of small molecules capable of reactivating p53 in RCC.

The same cells were used to screen a diverse library of 34,000 chemicals (see Materials and Methods) and 28 compounds that effectively activated p53 reporter were picked as primary hits. The most active compound, 30d9, was nine times stronger than doxorubicin (FIG. 1B), causing a 22-fold induction of the reporter. Structure-activity relationships established by the analysis of 59 structural analogues of 30d9 defined 9AA as the structure primarily responsible for induction of the reporter. 9AA induced stabilization and accumulation of p53 protein in treated cells (FIG. 6) accompanied by a dose-dependent increase in levels of the endogenous proteins p21 and Mdm2 that are encoded by p53-responsive genes (FIG. 1D). No increase in p53 mRNA expression was found (data not shown).

There were two known drugs among the derivatives of 9AA tested: the anti-cancer agent amsacrine (m-AMSA), an inhibitor of topoisomerase II, and the anti-malaria drug quinacrine (QC). Between these two, only QC was an effective inducer of p53 transactivation in RCC (FIG. 1C). All the biochemical and cellular effects described below were identical for 9AA and QC.

9AA activates p53-mediated transcription more strongly than DNA-damaging agents, not only in RCC but also in the majority of other types of tumor cells that have wild-type p53 (FIG. 1E). The strongest induction of p53 by 9AA was observed in human fibrosarcoma HT1080 cells, which were used in further assays along with RCC.

Activation of p53 response by 9AA had unusually slow kinetics. While the peak of doxorubicin-induced p53 reporter activity in HT1080 cells was at 8-12 h, the effect of 9AA on reporter activity as well as on the induction of endogenous p53 targets p21 and Mdm2 reached its maximum at 24 h (FIG. 1F,G).

Depending on the dose, 9AA caused either p53-dependent growth arrest or apoptosis (3 μM or 20 μM, respectively, FIG. 1H). Interestingly, low doses of 9AA did not cause apoptosis even after longer incubation times (up to 48 h), coinciding with the strongest induction of p21, while high doses of the compound induced apoptosis with no prior growth arrest, coinciding with the lack of p21 induction (FIGS. 1D,G,H). These effects were p53-dependent as evident by comparison with p53-deficient cells (FIG. 1H). Consistently, 9AA was less toxic to p53-deficient cells, as judged by analysis of a panel of isogenic pairs of cell lines differing in their p53 status due to shRNA-mediated p53 gene knockdown. Differential toxicity varied among the cell lines tested, with the maximal range observed in HT1080 cells (FIG. 1J and FIG. 6), possibly reflecting different degrees of p53 knockdown and/or the existence of a p53-independent mechanism of 9AA-mediated cell killing (see below). Importantly, the p53-dependence of cytotoxicity differentiated 9AA from camptothecin, doxorubicin, taxol and vinblastine, which were equally or even more toxic than 9AA to p53-deficient cells (FIG. 1K), suggesting that 9AA kills cells through a mechanism different from that utilized by conventional chemotherapeutic drugs.

RCC-derived cell lines were more sensitive to 9AA than normal kidney epithelial cells (FIG. 1L), consistent with the generally higher susceptibility of tumor cells to the cytotoxic effects of activated p53.

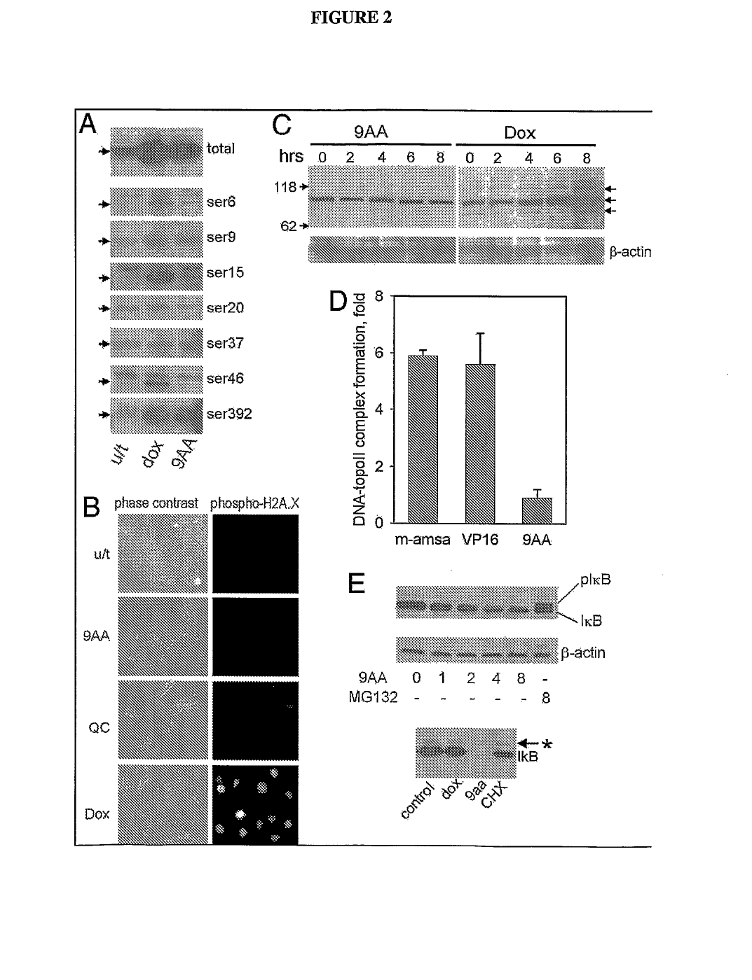

DNA damage is the most likely mechanism of p53 activation by chemicals and the DNA intercalating genotoxic activity was postulated as the basis of toxicity of 9AA and its derivatives (6, 7). However, n contrast to DNA-damaging drugs doxorubicin, 9AA and QC did not induce the phosphorylation of p53 (FIG. 2A); serine 392 that was increasingly phosphorylated after 9AA treatment is modified by protein kinase CK2 and not by DNA-damage-inducible kinases. Neither 9AA nor QC induced phosphorylation of histone H2A.X (FIG. 2B), a hallmark of DNA damage, nor did either activate the DNA breakage-dependent kinases ATM or ATR (FIG. 2C). While the 9AA derivative m-AMSA causes DNA damage by poisoning topoisomerase II (6), we found no such activity of 9AA in a direct in vitro assay (FIG. 2D). These observations indicated that 9AA activates p53 through a mechanism different from those activated by DNA damage.

p53 stabilization might be achieved by disrupting the binding of p53 to Mdm2 (8), a major mediator of p53 degradation. However, shRNA-mediated knockdown of Mdm2 did not lead to p53 reactivation in RCC (4), allowing us to exclude Mdm2 targeting as the mechanism of the p53-activating effect of 9AA.

The accumulation of unphosphorylated p53 suggested that 9AA might act as an inhibitor of proteasomal activity. This possibility was ruled out by using a direct in vitro assay (not shown) and by monitoring the effect of 9AA on the level of IκBα, another target of proteasomal degradation (9). Surprisingly 9AA treatment had the opposite effect opposite to the proteasomal inhibitor MG132, leading to a gradual decrease or even complete disappearance of IκBα (FIG. 2E).

### Example 2

### 9AA and QC Induce p53 by Inhibiting NF-κB

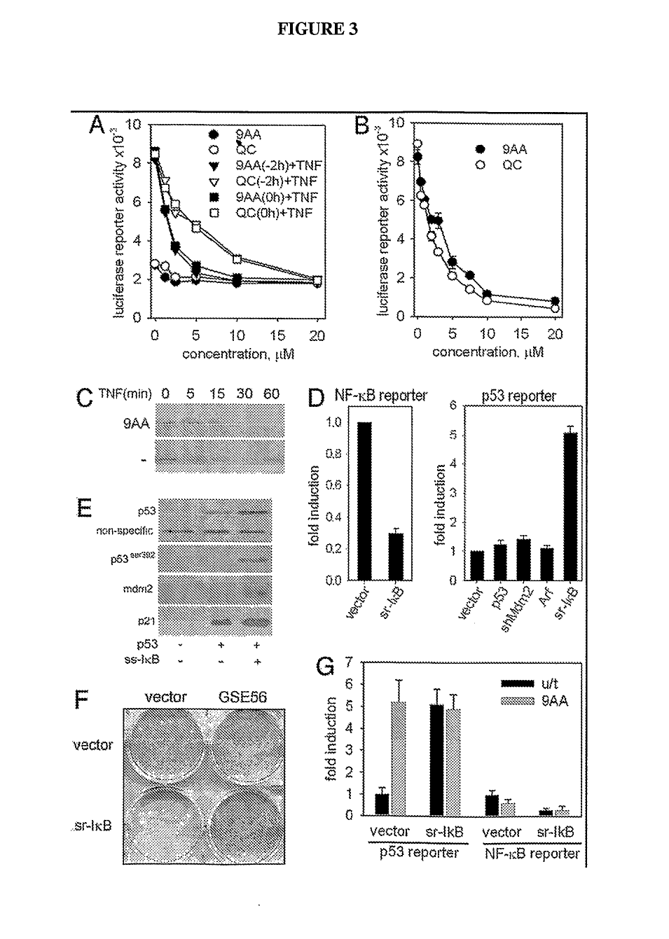

IκBα inhibits NF-κB by anchoring it in the cytoplasm; it is encoded by an NF-κB-inducible gene, acting as part of a negative feedback regulatory loop (10). The decrease in IκBα in 9AA-treated cells could be explained either by increased degradation of the protein or by inhibition of NF-κB-dependent transcription. To distinguish between these possibilities, we monitored NF-κB-response in the cells treated with different concentrations of compounds in the presence or absence of the NF-κB-inducing cytokine TNF. Both 9AA and QC showed a strong dose-dependent suppression of basal and TNF-induced NF-κB reporter activities (FIG. 3A) and blocked TNF-stimulated induction of IκBα (FIG. 3B). This effect was quite specific, since 9AA and QC did not change the activity of several other transcriptional activators, including the androgen receptor, N-Myc, CLOCK/Bmal and Smad (data not shown). The results of global gene expression profiling by microarray hybridization confirmed the dual effect of 9AA as an inducer of p53 and an inhibitor of NF-κB transcription (see Materials and Methods and Tables 1 and 2).

Are the effects of 9AA on p53 activation and NF-κB repression related or distinct activities of the drug, and, if interrelated, which is the primary event? We could readily exclude the possibility that p53 activation drives NF-κB repression since all the effects of 9AA on the NF-κB pathway were seen in p53-deficient (H1299, PC3) as well as in p53 wild type (HT1080, RCC) cells. To explore the alternative model (NF-κB repression by 9AA drives p53 activation) we analyzed p53 activity in the cells with NF-κB inhibited by genetic approach. To suppress NF-κB activity, we used IκB super-repressor (sr-IκB), a stable IκB mutant lacking both phosphorylation sites (9). Transduction of this mutant into RCC ACHN cells resulted in a three-fold inhibition of NF-κB reporter activity (FIG. 3D), consistent with the constitutive activity of NF-κB in RCC cell lines (11). Importantly, the activity of a p53-responsive reporter was increased up to five times upon transduction of sr-IκB (FIG. 3D). A similar effect was observed in another RCC line, RCC54, as well as in non-RCC HT1080 cells (data not shown). Remarkably, sr-IκB activated p53 in RCC while such direct regulators of p53 activity, as Arf, shRNA to Mdm2 or p53 itself failed to do it (FIG. 3D). Moreover, ectopic expression of sr-IκB led to the accumulation of p53, a consequent increase in p21 and Mdm2, and to an increase in p53 phosphorylated at serine residue 392, all seen after 9AA treatment (FIGS. 3E and 2A).

Similarly to treatment with 9AA, ectopic expression of sr-IκB was toxic to HT1080 and RCC45 cells, reflecting their addiction to constitutively active NF-kB, and, again similarly to 9AA, this toxicity was p53-dependent and was greatly reduced by expression of either anti-p53 shRNA or by of the dominant-negative p53 mutant protein GSE56 (FIG. 3F). These results provide a plausible explanation for otherwise puzzling reports indicating resistance to apoptosis of cells selected for sr-IκB expression (12),(13). This resistance probably resulted from inactivation of p53 function.

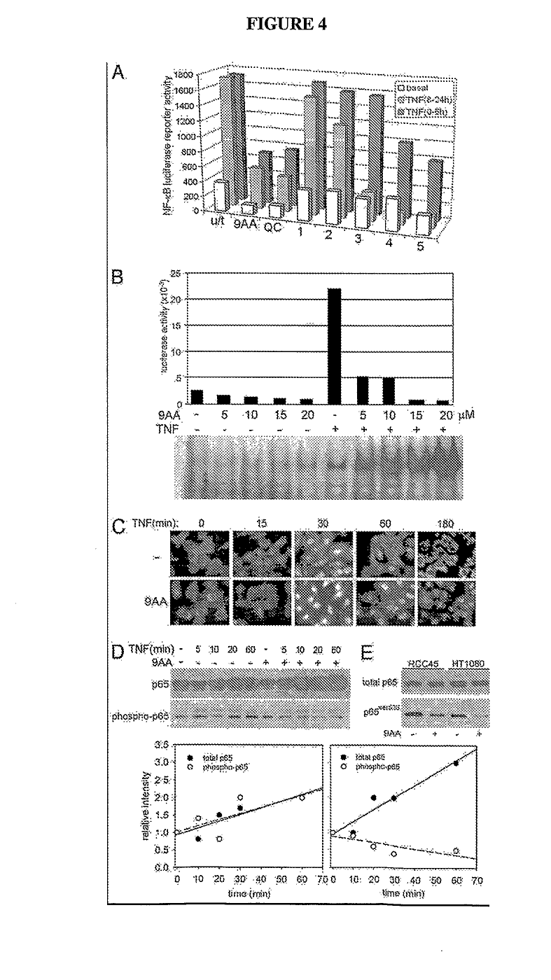

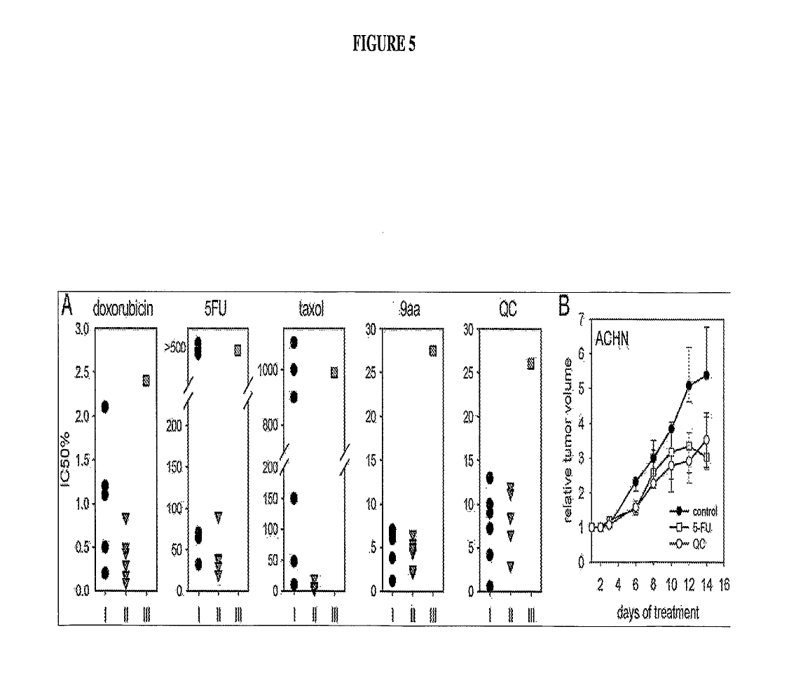

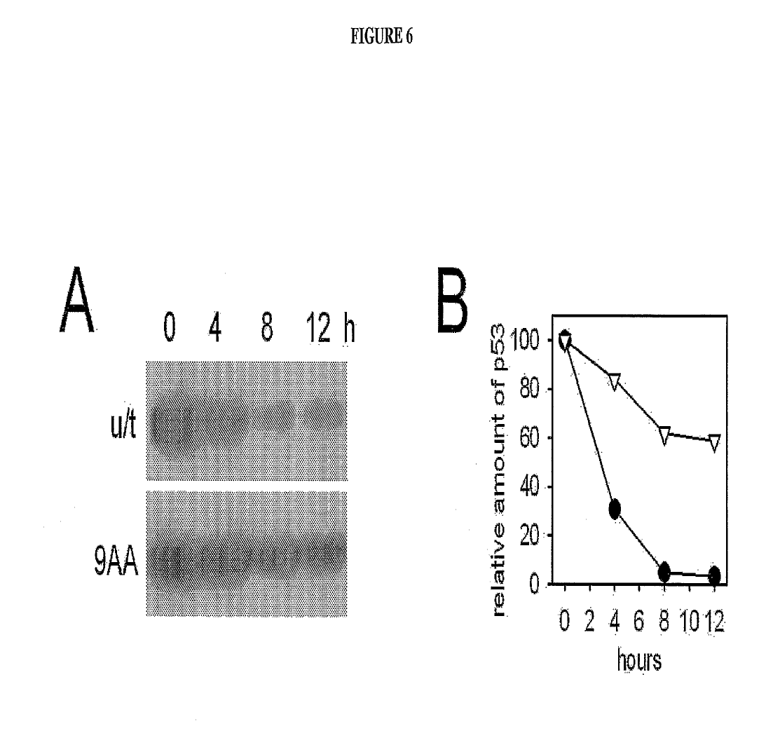

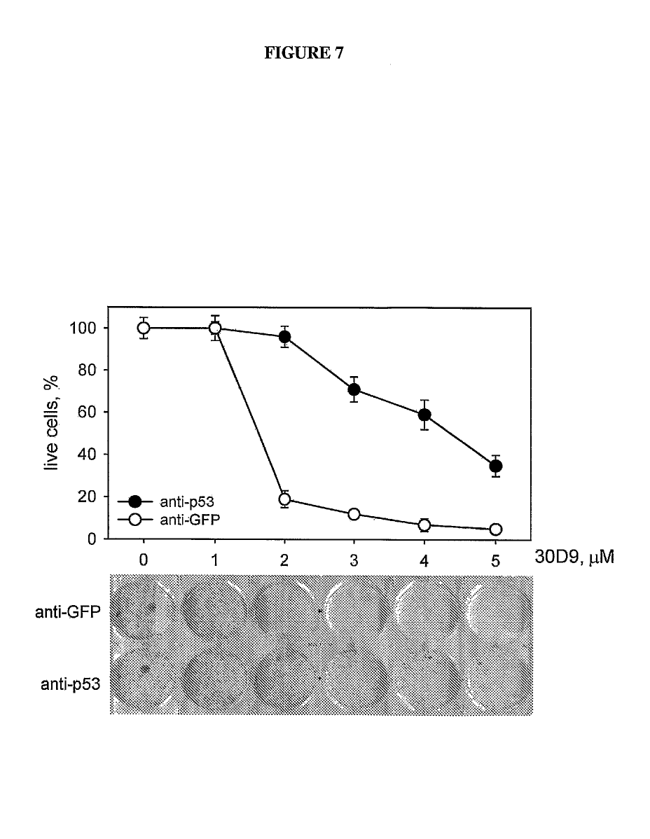

Since stable expression of sr-IκB interfered with RCC cell viability, the effect of 9AA on cells with repressed NF-κB was tested in transient transfection experiments, in which the introduction of sr-IκB was combined with either NF-κB- or p53-responsive reporters. 9AA could not further activate a p53-dependent reporter in the presence of sr-IκB (FIG. 4G), indicating that its p53-activating effect is indeed mediated through inhibition of NF-κB and that NF-κB activity is responsible for the repression of p53 activity in RCC and other types of tumor cells.

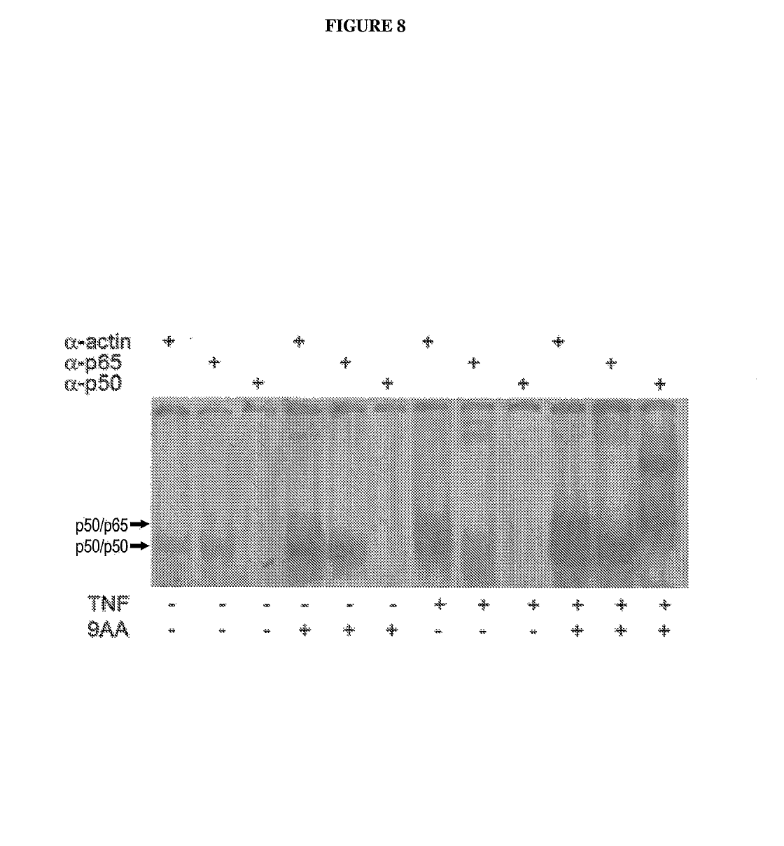

The only known mechanism of mutual negative regulation of p53 and NF-κB is their competition for CBP/p300 transcriptional coactivators (14). We overexpressed CBP/p300 ectopically and traced their effects on p53 activity in HT1080 and ACHN cells, alone and in combination with sr-IκB (FIG. 8), concluding that the NF-κB-mediated inhibition of p53 activity in these cells cannot be explained by depletion of transcriptional coactivators and must be due to another mechanism. The dominant nature of p53 repression (4) and the unusually slow kinetics of p53 de-repression upon NF-κB inhibition in RCC suggest that the NF-κB-mediated p53 repression is due to an inhibitory factor encoded by an NF-κB-responsive gene, which is depleted when NF-κB is not active. It is likely that identification of their direct molecular target(s) of 9AA and QC will help to uncover new important regulatory components of NF-κB- and p53-dependent signaling that provide interactions between these two major stress-responsive pathways.

### Example 3

### 9AA and QC Represent a New Type of NF-κB Inhibitor

Since NF-κB appeared to be the primary target of 9AA, we focused on the mechanism of 9AA-mediated NF-κB suppression. 9AA and QC were capable of inhibiting both basal and TNF-induced activities of NF-κB in H1299 (FIG. 4A, B), RCC45 and RCC54 (Table 2 in Supporting Materials) and other cells. Inhibition of the basal activity of NF-κB is a unique property of 9AA compounds, as known NF-κB inhibitors, sulfasalazine (15), capsaicin (16) and Bay11-7082 (17), tested side-by-side with 9AA, did not reduce the basal activity of an NF-κB (FIG. 4A), what explains why they were capable of activating p53 (data not shown). Moreover, 9AA and QC can inhibit NF-κB when added before, simultaneously with or after TNF stimulation (FIGS. 3A and 4A), while known NF-κB inhibitors required several hours of pretreatment to be effective.

Another distinction between 9AA and other NF-κB inhibitors was its paradoxical effect on the DNA binding by NF-κB. Simultaneously with the inhibition of TNF-stimulated NF-κB-dependent transcription, 9AA caused a significant increase in the binding of NF-κB to DNA that correlated with the nuclear accumulation of p65-containing NF-κB complexes (FIG. 4B). As opposed to NF-κB inhibitors that lock transcription complexes in the cytoplasm by inhibiting the phosphorylation and degradation of IκBα (18),(19),(10), 9AA and QC not only allowed the TNF-induced nuclear translocation and accumulation of NF-κB but greatly prolonged the time of its presence in nuclei (FIG. 4C). Thus, 9AA and QC act by converting NF-κB to a transcriptionally inactive state that becomes trapped in the nucleus. This mechanism seems to be more relevant to anti-inflammatory properties of QC, which was used previously to treat rheumatoid disease, than the inhibition of phospholipase A2 (PLA2), traditionally considered to be the QC target (20). It is noteworthy that other PLA2 inhibitors we tested (thioetheramide-PC, arachidonyl trifluoromethyl ketone and others) could neither induce p53 nor inhibit NF-κB (data not shown).

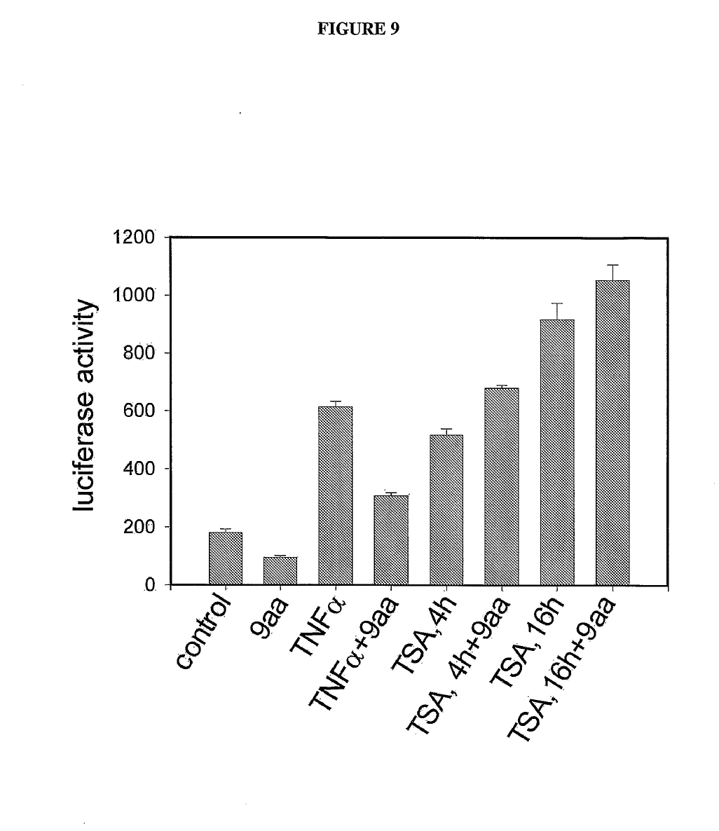

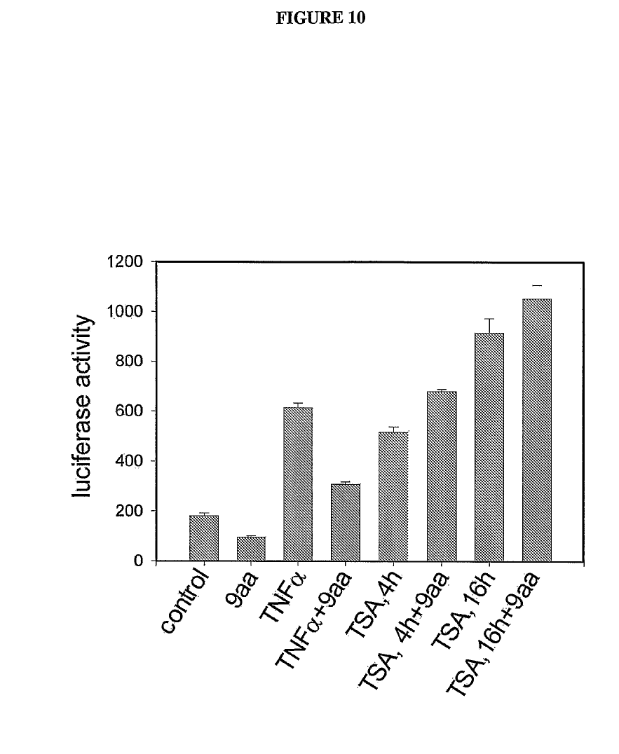

Several factors can affect the activity of nuclear NF-κB, including composition of the complex (the stoichiometry of p65 and p50 subunits, the presence of transcriptional co-activators or co-repressors, etc.), post-translational modifications of components of the complexes (i.e., phosphorylation or acetylation of p65) or modification of histones (i.e., deacetylation or phosphorylation) in chromatin near sites of initiation of NF-κB-dependent transcription (5). While treatment with 9AA did not significantly change the composition of NF-κB complexes, as judged by gel-shift assays using antibodies to different components (FIG. 9), it did reduce the proportion of phosphorylated serine 536 in the p65 subunit of NF-κB, both basally and after TNF-dependent induction (FIG. 4D,E). This modification of p65 by IκB kinase 1 or other kinases is essential for the NF-κB activity in several cell systems and lack of this phosphorylation can convert NF-κB into a transrepressor that acts by recruiting histone deacetylases (HDAC) (21),(22). Remarkably, this modification of p65 was reported to be responsible for inhibition of p53 in HTLV-infected T-lymphocytes, mediated by the tax protein (23, and references therein). These observations suggest that inhibition of serine 536 phosphorylation could be the primary mechanism of 9AA-mediated inhibition of NF-κB. In accordance with this model, the inhibition of HDAC activity by trichostatin A (TSA) resulted in a strong activation of NF-κB-dependent transcription that could no longer be blocked by 9AA (FIG. 10). Serine 536 is phosphorylated by numerous kinases, including IκB kinase 1, NIK, TBK1, PKC, PKA (24), each of which could be the target for 9AA. For example, IκB kinase 1 is responsible for basal NF-κB activity in several cell types (25) and is involved in the control of NF-κB activity at the promoter level (26). Moreover, a deficit in IκB kinase 1 activity increases the half-life of p65 in nuclei (27). All these traits are affected by 9AA treatment, making this kinase a primary suspect for being the 9AA target.

### Example 4

### Perspectives of Anti-Tumor Applications of QC

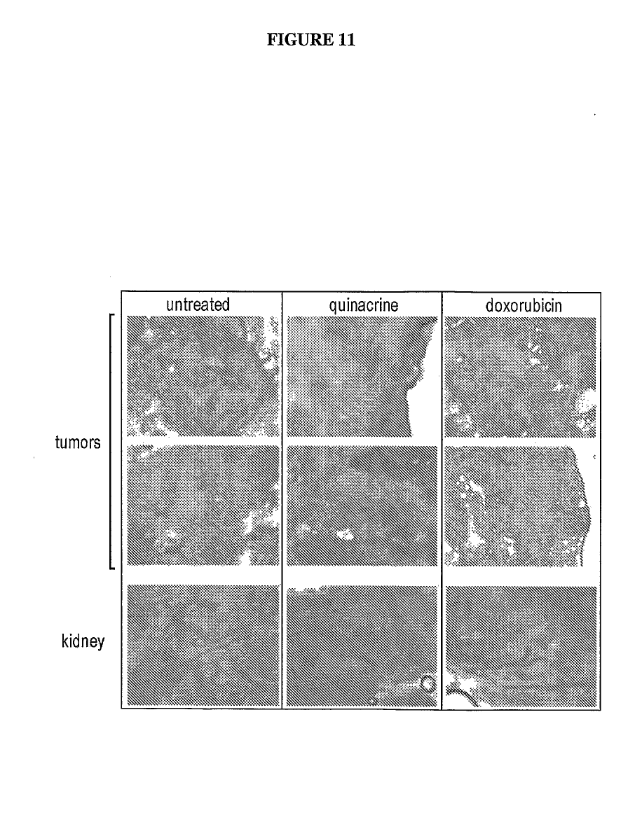

Since RCC is one of the most drug-resistant tumors, we assessed whether 9AA-based compounds would be more potent in killing RCC cells than conventional chemotherapeutic agents, by comparing doxorubicin, taxol and 5-fluorouracil with 9AA and QC in a set of RCC and non-RCC-derived tumor cells (6 of each type), as well as in NKE. The average IC50 in RCC was higher than in non-RCC cells for all chemotherapeutic agents used, and near the IC50 of normal cells. However, the IC50s of 9AA and QC for RCC cells were in the same range as for non-RCC cells (FIG. 6A). Moreover, QC inhibited the growth of tumor xenografts formed by subcutaneous injection of ACHN cells into nude mice. QC, showing the same antitumor effect as 5-fluorouracil, but without the significant weight loss (up to 20%) that accompanied treatment with this drug (FIG. 6B). We also noted that the p53-inducing activity of QC is not limited to tumor cells in culture but can be also detected in short-term organ cultures of surgically removed human RCCs (see FIG. 11).

In conclusion, 9AA can be viewed as the prototype of a new class of bi-targeted anticancer drugs that attack simultaneously and in the desirable direction two important stress responsive pathways. The ability to simultaneously inhibit NF-κB and activate p53 makes 9AA-based compounds potentially useful against tumors which, like RCC, maintain wild-type p53 in a state that is completely or partially repressed by constitutively active NF-κB (28),(11),(29). Presence of QC among these compounds, the drug with a long history of broad human use as an anti-malaria and anti-arthritis agent with favorable pharmacological and toxicological properties (30), opens the opportunity of rapid clinical evaluation of this approach.

### Example 5

### Modulation of the Immune Response

Constitutively active NF-κB signaling, an attribute of chronic inflammation and the property of many tumors, provides selective advantages to tumor cells, probably by inhibiting apoptosis and promoting proliferation by stimulating expression of anti-apoptotic factors and cytokines (5). Above, we show that inhibition of tumor suppressor p53 is another important benefit to tumors that have constitutively active NF-κB, opening the possibility of simultaneous inhibition of NF-κB and activation of p53 by a single small molecule. Among the compounds effective at inhibiting NF-κB were derivatives of 9-aminoacridine (9AA), including an old-known anti-malaria drug quinacrine (QC). Analysis of the molecular mechanisms of action of 9AA and QC showed that p53 activation by these compounds occurred through the inhibition of constitutively active NF-κB in tumor cells.

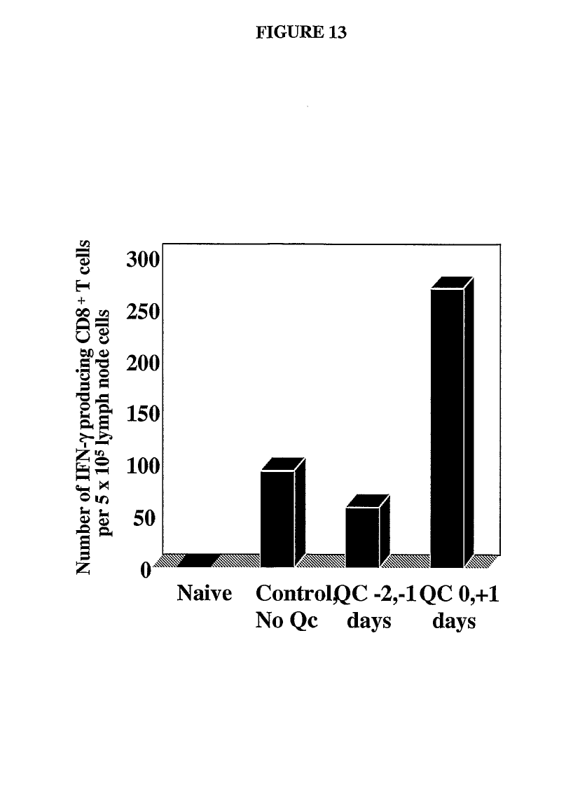

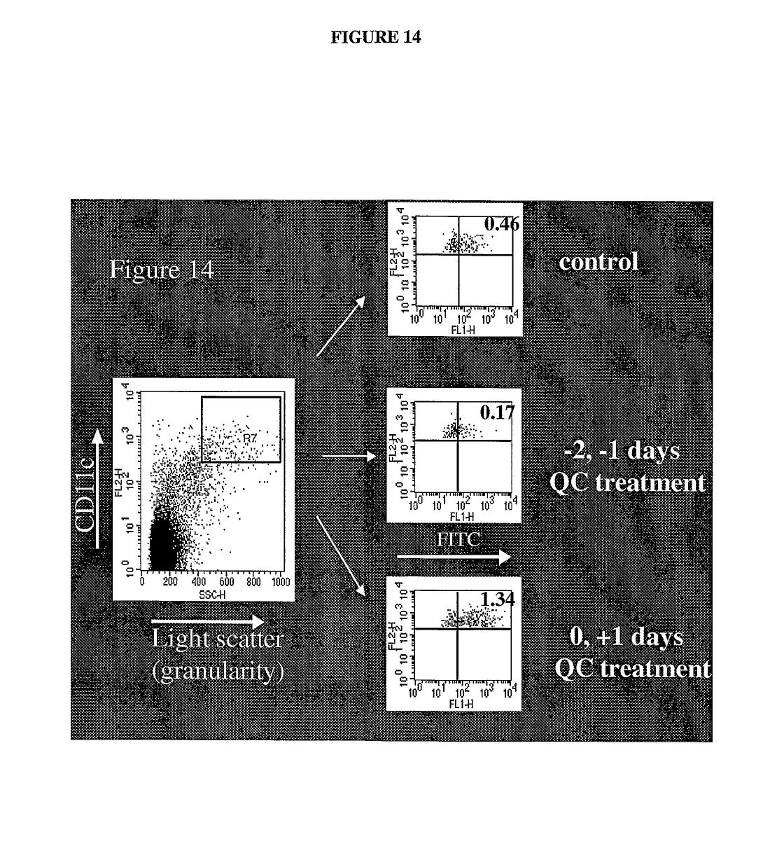

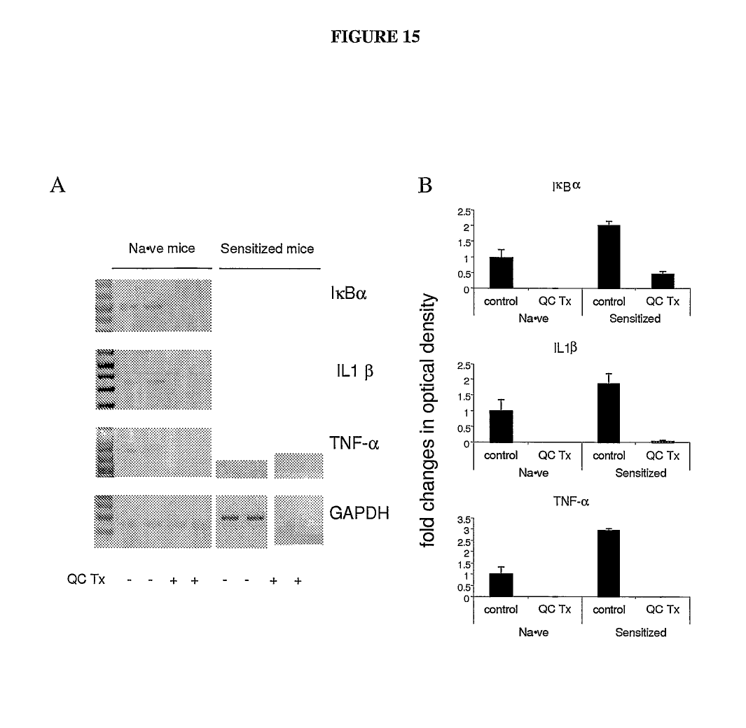

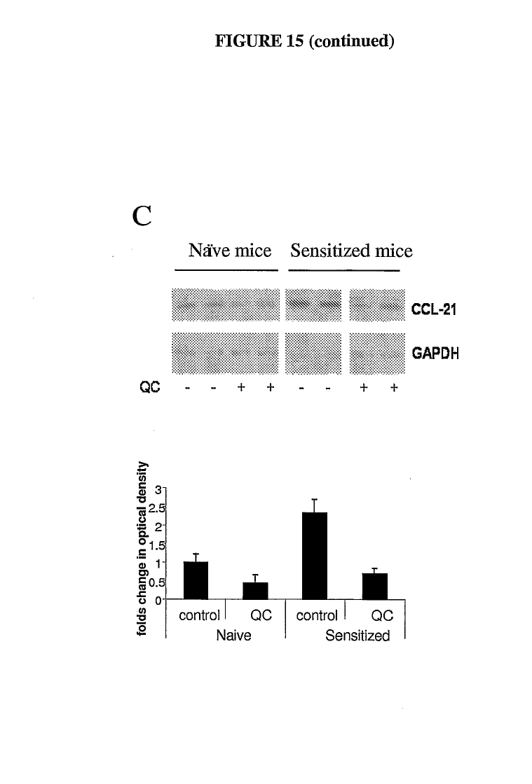

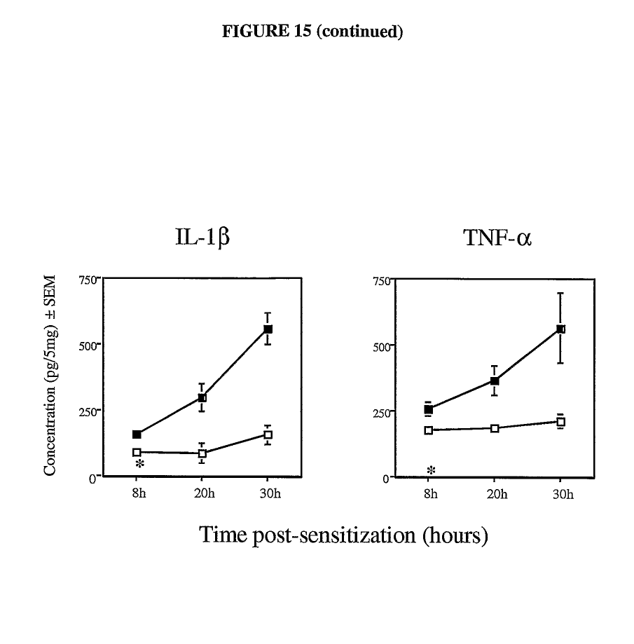

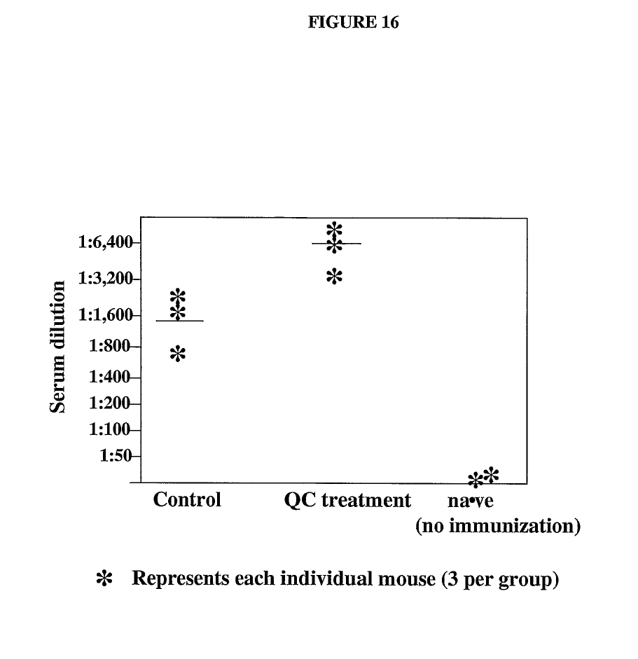

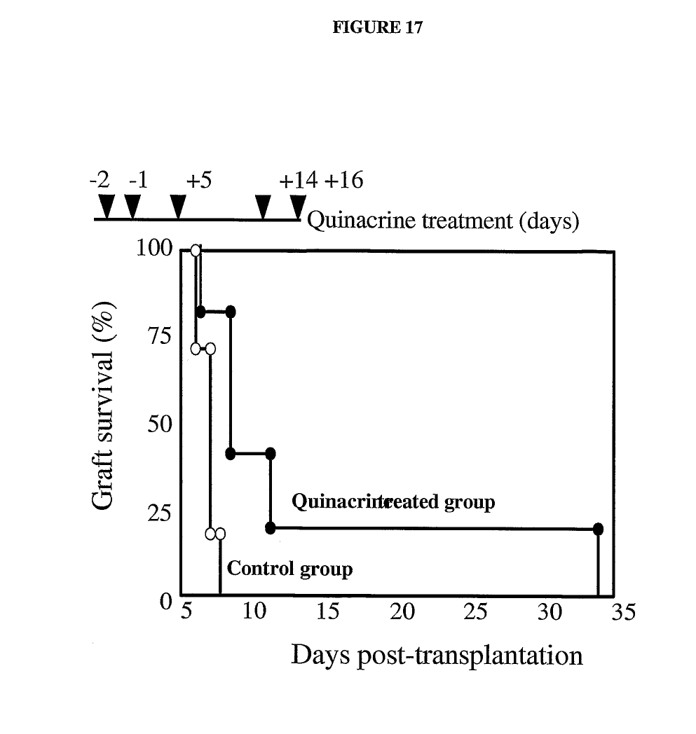

Based on the role of NF-κB activity in the immune response, we tested whether the NF-κB inhibiting compounds could also modulate the immune response. Our results indicate that the administration of quinacrine during antigen sensitization of mice for T cell mediated contact hypersensitivity responses inhibits the priming of antigen-specific T cells (FIG. 13) and the magnitude and duration of the allergic response elicited (FIG. 12). Furthermore, pretreatment with quinacrine to recipients of heterotopically transplanted MHC-mismatched cardiac allografts in a murine model resulted in significant prolongation of allograft survival (FIG. 17). The data also indicated that administration of quinacrine after antigen sensitization increased the magnitude and persistence of the antigen-specific T cell response (FIG. 12).

TABLE 1
List of known p53-inducible genes upregulated by 9AA treatment
in a dose-dependent manner in both RCC45 and RCC54 cells

| | Relative expression compared | | | | |
|---|---|---|---|---|---|
| | to untreated cells | | | | |
| | RCC54 | | RCC45 | | |
| gene | 2 μM | 10 μM | 2 μM | 10 μM | references |
| Cyclin-dependent kinase inhibitor 1A | 1.25 | 3.80 | 1.33 | 4.62 | Rev. by Nakamura. 2004. |
| (p21, Cip1) | | | | | Cancer Sci. 95: 7 |
| Mdm2, transformed 3T3 cell double | 0.96 | 2.86 | 0.91 | 2.30 | |
| minute 2, p53 binding protein (mouse) | | | | | |
| Growth arrest and DNA-damage- | 1.22 | 2.78 | 0.92 | 2.66 | |
| inducible, beta | | | | | |
| Ribonucleotide reductase M2 B (TP53 | 1.11 | 3.53 | 1.08 | 3.33 | Ceballos E, et al. Oncogene. |
| inducible) | | | | | 2005 Apr 18; |
| Annexin A1 | 1.01 | 3.04 | 1.13 | 2.02 | Kannan et al. 2001. |
| | | | | | Oncogene 20: 3449 |
| Leucine-rich repeats and death domain | 1.12 | 2.12 | 1.10 | 1.64 | Lin et al. 2000. Nat Genet. |
| containing | | | | | 26: 122 |
| Tumor protein p53 inducible nuclear | 1.03 | 1.61 | 1.09 | 1.86 | Okamura et al. 2001. Mol. |
| protein 1 | | | | | Cell. 8: 85 |
| Apoptotic protease activating factor | 0.78 | 1.27 | 0.85 | 1.72 | Fortin et al. 2001. J. Cell. |
| | | | | | Biol. 155: 207 |
| Heat shock 27 kDa protein 1 | 0.82 | 1.30 | 1.13 | 1.71 | Gao et al. 2000. Int. J. |
| | | | | | Cancer. 88: 191 |
| KILLER/DR5 | 1.1 | 1.9 | 1.1 | 2.4 | Takimoto and El-Deiry |
| | | | | | 2000. Oncogene 19: 1735 |
| BCL2 binding component 3 (PUMA) | 1.03 | 1.57 | 0.78 | 1.79 | Nakano and Vousden. |
| | | | | | 2001. Mol. Cell. 7: 683 |
| Damage-specific DNA binding protein 2, | 0.89 | 1.38 | 1.00 | 1.79 | Kannan et al. Oncogene. |
| 48 kDa | | | | | 2001. 20: 2225 |
| Carboxylesterase 2 (intestine, liver) | 0.80 | 1.75 | 1.04 | 3.17 | |
| Thiosulfate sulfurtransferase (rhodanese) | 1.18 | 3.59 | 1.13 | 2.79 | |
| Amyloid beta (A4) precursor-like protein | 1.19 | 1.68 | 0.97 | 1.90 | |
| 2 | | | | | |
| Activating transcription factor 3 | 1.07 | 3.56 | 0.91 | 2.99 | |
| BTG family member 2 | 1.44 | 1.88 | 0.9 | 2.4 | |

TABLE 2
List of known NF-kB-inducible genes downregulated by 9AA treatment
in a dose-dependent manner in both RCC45 and RCC54 cells

| | Relative expression compared | | | | |
|---|---|---|---|---|---|
| | to untreated cells | | | | |
| | RCC54 | | RCC45 | | |
| gene | 2 μM | 10 μM | 2 μM | 10 μM | references |
| Chemokine (C-X-C motif) ligand 1 | 0.40 | 0.08 | 0.93 | 0.15 | Loukinova et al. Int J |
| (melanoma growth stimulating activity, | | | | | Cancer. 2001. 94: 637 |
| alpha) | | | | | |
| Interleukin 8 | 0.62 | 0.09 | 1.37 | 0.30 | Kunsch et al. J Immunol. |
| | | | | | 1994. 153: 153 |
| Chemokine (C-X-C motif) ligand 6 | 1.22 | 0.11 | 0.94 | 0.22 | Loukinova et al. Int J |
| (granulocyte chemotactic protein 2) | | | | | Cancer. 2001. 94: 637 |
| Chemokine (C-C motif) ligand 20 | 0.64 | 0.26 | 1.06 | 0.35 | Carson et al. Cancer Res. |
| | | | | | 2004. 64: 2096 |
| Tenascin C (hexabrachion) | 0.70 | 0.59 | 1.37 | 0.58 | Mettouchi et al. Mol Cell |
| | | | | | Biol. 1997. 17: 3202 |
| Nuclear factor of kappa light polypeptide | 0.73 | 0.54 | 0.96 | 0.55 | Hinz et al. J Exp Med. |
| gene enhancer in B-cells inhibitor, alpha | | | | | 2002. 196: 605 |
| Cyclin D1 (PRAD1: parathyroid | 0.77 | 0.29 | 1.34 | 0.69 | Guttridge et al. Mol Cell |
| adenomatosis 1) | | | | | Biol. 1999. 19: 5785 |
| V-myc myelocytomatosis viral oncogene | 1.19 | 0.40 | 0.88 | 0.48 | Bourgarel-Rey et al. Mol |
| homolog (avian) | | | | | Pharmacol. 2001. 59: 1165 |
| Chemokine-like factor super family 3 | 0.87 | 0.56 | 0.85 | 0.59 | Mettouchi et al. Mol Cell |
| Tumor necrosis factor, alpha-induced | 1.26 | 0.48 | 0.73 | 0.27 | Biol. 1997. 17: 3202 |
| protein 6 | | | | | |
| Tumor necrosis factor receptor | 0.63 | 0.85 | 0.65 | 0.91 | Kim et al. FEBS Lett. 2003. |
| superfamily, member 9 | | | | | 541: 163 |
| Vascular cell adhesion molecule 1 | 0.88 | 0.53 | 1.18 | 0.51 | Iademarco et al. J Biol |
| | | | | | Chem. 1992. 267: 16323 |
| Chemokine (C-C motif) ligand 2 | 0.64 | 0.26 | 1.06 | 0.35 | Martin et al. Eur J Immunol. |
| | | | | | 1997. 27: 1091 |
| CD44 antigen (homing function and Indian | 0.88 | 0.65 | 1.32 | 0.41 | Hinz et al. J Exp Med. |
| blood group system) | | | | | 2002. 196: 605 |

### REFERENCES

1. Prives, C. & Hall, P. A. (1999) J Pathol 187, 112-26.
2. Olivier, M., Hussain, S. P., Caron de Fromentel, C., Hainaut, P. & Harris, C. C. (2004) IARC Sci Publ, 247-70.
3. Gudkov, A. V. (2005) in The p53 Tumor Suppressor Pathway and Cancer, ed. Zambetti, G. (Springer, Vol. 2.
4. Gurova, K. V., Hill, J. E., Razorenova, O. V., Chumakov, P. M. & Gudkov, A. V. (2004) Cancer Res 64, 1951-8.
5. Orlowski, R. Z. & Baldwin, A. S., Jr. (2002) Trends Mol Med 8, 385-9.
6. Zwelling, L. A., Hinds, M., Chan, D., Mayes, J., Sie, K. L., Parker, E., Silberman, L., Radcliffe, A., Beran, M. & Blick, M. (1989) J Biol Chem 264, 16411-20.
7. Sohn, T. A., Bansal, R., Su, G. H., Murphy, K. M. & Kern, S. E. (2002) Carcinogenesis 23, 949-57.
8. Vassilev, L. T., Vu, B. T., Graves, B., Carvajal, D., Podlaski, F., Filipovic, Z., Kong, N., Kammlott, U., Lukacs, C., Klein, C., Fotouhi, N. & Liu, E. A. (2004) Science 303, 844-8.
9. Baldi, L., Brown, K., Franzoso, G. & Siebenlist, U. (1996) J Biol Chem 271, 376-9.
10. Panwalkar, A., Verstovsek, S. & Giles, F. (2004) Cancer 100, 1578-89.
11. Oya, M., Takayanagi, A., Horiguchi, A., Mizuno, R., Ohtsubo, M., Marumo), K., Shimizu, N. & Murai, M. (2003) Carcinogenesis 24, 377-84.
12. Ryan, K. M., O'Prey, J. & Vousden, K. H. (2004) Cancer Res 64, 4415-8.
13. Fujioka, S., Sclabas, G. M., Schmidt, C., Niu, J., Frederick, W. A., Dong, Q. G., Abbruzzese, J. L., Evans, D. B., Baker, C. & Chiao, P. J. (2003) Oncogene 22, 1365-70.
14. Webster, G. A. & Perkins, N. D. (1999) Mol Cell Biol 19, 3485-95.
15. Muerkoster, S., Arlt, A., Witt, M., Gehrz, A., Haye, S., March, C., Grohmann, F., Wegehenkel, K., Kalthoff, H., Folsch, U. R. & Schafer, H. (2003) Int J Cancer 104, 469-76.
16. Kim, C. S., Kawada, T., Kim, B. S., Han, I. S., Choe, S. Y., Kurata, T. & Yu, R. (2003) Cell Signal 15, 299-306.
17. Cahir-McFarland, E. D., Carter, K., Rosenwald, A., Giltnane, J. M., Henrickson, S. E., Staudt, L. M. & Kieff, E. (2004) J Virol 78, 4108-19.
18. Lenz, H. J. (2003) Cancer Treat Rev 29 Suppl 1, 41-8.
19. Greten, F. R. & Karin, M. (2004) Cancer Lett 206, 193-9.
20. Al Moutaery, A. R. & Tariq, M. (1997) Digestion 58, 129-37.
21. Zhong, H., May, M. J., Jimi, E. & Ghosh, S. (2002) Mol Cell 9, 625-36.
22. Zhang, W. & Kone, B. C. (2002) Am J Physiol Renal Physiol 283, F904-11.
23. Pise-Masison, C. A. & Brady, J. N. (2005) Front Biosci 10, 919-30.
24. Viatour, P., Merville, M. P., Bours, V. & Chariot, A. (2005) Trends Biochem Sci 30, 43-52.
25. Li, X., Massa, P. E., Hanidu, A., Peet, G. W., Aro, P., Savitt, A., Mische, S., Li, J. & Marcu, K. B. (2002) J Biol Chem 277, 45129-40.
26. Yamamoto, Y., Verma, U. N., Prajapati, S., Kwak, Y. T. & Gaynor, R. B. (2003) Nature 423, 655-9.
27. Lawrence, T., Bebien, M., Liu, G. Y., Nizet, V. & Karin, M. (2005) Nature 434, 1138-43.
28. Oya, M., Ohtsubo, M., Takayanagi, A., Tachibana, M., Shimizu, N. & Murai, M. (2001) Oncogene 20, 3888-96.
29. Nikolaev, A. Y., Li, M., Puskas, N., Qin, J. & Gu, W. (2003) Cell 112, 29-40.
Wallace, D. J. (1989) Semin Arthritis Rheum 18, 282-96.
1a. Gurova, K. V., Hill, J. E., Razorenova, O. V., Chumakov, P. M. & Gudkov, A. V. (2004) Cancer Res 64, 1951-8.
2a. Brummelkamp, T. R., Bernards, R. & Agami, R. (2002) Science 296, 550-3.
3a. Gurova, K. V., Kwek, S. S., Koman, I. E., Komarov, A. P., Kandel, E., Nikiforov, M. A. & Gudkov, A. V. (2002) Cancer Biol Ther 1, 39-44.
4a. Aoyama, M., Grabowski, D. R., Dubyak, G. R., Constantinou, A. I., Rybicki, L. A., Bukowski, R. M., Ganaphthi, M. K., Hickson, I. D. & Ganaphthi, R. (1998) Biochem J 336 (Pt 3), 727-33.
5a. Ganaphthi, R., Constantinou, A., Kamath, N., Dubyak, G., Grabowski, D. & Krivacic, K. (1996) Mol Pharmacol 50, 243-8.
6a. Zwelling, L. A., Hinds, M., Chan, D., Mayes, J., Sie, K. L., Parker, E., Silberman, L., Radcliffe, A., Beran, M. & Buick, M. (1989) J Biol Chem 264, 16411-20.
7a. Chemov, M. V. & Stark, G. R. (1997) Oncogene 14, 2503-10.

1. A method of enhancing, inhibiting or suppressing a mammal's immune response, comprising administering to a mammal in need thereof an aminoacridine of the formula: 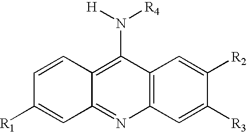
wherein,
R₁ is H or halogen;
R₂ is H or optionally substituted alkoxy;
R₃ is H or optionally substituted alkoxy; and
R₄ is H or optionally substituted aliphatic, aryl, or heterocycle.

2. The method of claim 1, wherein the aminoacridine is selected from the group consisting of 9-aminoacridine and quinacrine.

3. The method of claim 1, wherein the mammal is selected from the group consisting of clinically normal and immunodeficient subjects, subjects afflicted by auto-immune conditions, and transplant subjects.

4. A method of boosting the immune response of a mammal to a vaccine, comprising:
(a) enhancing the immune response of the mammal according to the method of claim 1; and
(b) administering a vaccine to the mammal.


SEQUENCE LISTING (10 sequences)

SEQ ID NO: 1 - DNA, 23 bp, Mus musculus
tcgtggatctgacgtgccgcctg

SEQ ID NO: 2 - DNA, 25 bp, Mus musculus
caccaccctgttgctgtagccgtat

SEQ ID NO: 3 - DNA, 20 bp, Mus musculus
ctgggagctggctgtgatcc

SEQ ID NO: 4 - DNA, 21 bp, Mus musculus
tctgtgtcatagctctcctca

SEQ ID NO: 5 - DNA, 22 bp, Mus musculus
aaatgcctcgtgctgtctgacc

SEQ ID NO: 6 - DNA, 24 bp, Mus musculus
ctgcttgagaggtgctgatgtacc

SEQ ID NO: 7 - DNA, 24 bp, Mus musculus
atgagcacagaaagcatgatccgc

SEQ ID NO: 8 - DNA, 23 bp, Mus musculus
ccaaagtagacctgcccggactc

SEQ ID NO: 9 - DNA, 20 bp, Mus musculus
caggactgctgccttaagta

SEQ ID NO: 10 - DNA, 20 bp, Mus musculus
gcacatagctcaggcttaga